(19) United States
(12) Patent Application Publication
(10) Pub. No.: US 2026/0026734 A1
(43) Pub. Date: Jan. 29, 2026

(54) DETECTION OF CRANIAL ACUTE HEALTH EVENT

(71) Applicant: Covidien LP, Mansfield, MA (US)

(72) Inventors: Randal C. Schulhauser, Phoenix, AZ (US); Tarek D. Haddad, Minneapolis, MN (US); Merdim Sonmez, Irvine, CA (US); Ekaterina B. Morgounova, Minneapolis, MN (US); Ashwini D. Sharan, Pennington, NJ (US)

(21) Appl. No.: 19/270,946

(22) Filed: Jul. 16, 2025

Related U.S. Application Data
(60) Provisional application No. 63/675,624, filed on Jul. 25, 2024.

(51) Int. Cl. A61B 5/374; A61B 5/00; G16H 50/20

(52) U.S. Cl. CPC A61B 5/374; A61B 5/7264; G16H 50/20

(57) ABSTRACT

An example system includes a plurality of electrodes; sensing circuitry configured to: generate, based on sensed electrical signals, one or more electroencephalography (EEG) signals; and processing circuitry configured to: receive one or more EEG signals generated during a first period of time; determine, based on at least one particular feature of the one or more EEG signals generated during the first period of time, an initial indication of a cranial acute health event during the first period of time; receive, from the sensing circuitry, one or more EEG signals generated during a second period of time; determine whether a difference between the at least one particular feature of the one or more EEG signals generated during the first period of time and at least one corresponding particular feature of the one or more EEG signals generated during the second period of time satisfies a cranial acute health event mimic threshold.

### CROSS-REFERENCE TO RELATED APPLICATIONS

This application claims the benefit of and priority to U.S. Provisional Patent Application Ser. No. 63/675,624 filed Jul. 25, 2024, the entire disclosure of which is incorporated by reference herein.

### TECHNICAL FIELD

This disclosure is directed to medical devices and, more particularly, to systems and methods for detecting a cranial acute health event.

### BACKGROUND

Stroke is a serious medical condition that can cause permanent neurological damage, complications, and death. Stroke may be characterized as the rapidly developing loss of brain functions due to a disturbance in the blood vessels supplying blood to the brain. The loss of brain functions can be a result of ischemia (lack of blood supply) caused by thrombosis or embolism, or hemorrhage (e.g., a ruptured blood vessel). During a stroke, the blood supply to an area of a brain may be decreased, which can lead to dysfunction of the brain tissue in that area.

Stroke is the number two cause of death worldwide and the number one cause of disability. Speed to treatment is the critical factor in stroke treatment as 1.9M neurons are lost per minute on average during stroke. Stroke diagnosis and time between event and therapy delivery are the primary barriers to improving therapy effectiveness. Stroke has 3 primary etiologies; i) ischemic stroke (representing approximately 65% of all strokes), ii) hemorrhagic stroke (representing approximately 10% of all strokes), and iii) cryptogenic strokes (includes TIA, representing approximately 25% of all strokes). In an ischemic stroke, a blood clot occludes blow flow in an artery within the brain. In a hemorrhagic stroke, a blood vessel bursts within the brain. Strokes can be considered as having neurogenic and/or cardiogenic origins.

A variety of approaches exist for treating patients undergoing a stroke. For example, a clinician may administer anticoagulants, such as warfarin, or may undertake intravascular interventions such as thrombectomy procedures to treat ischemic stroke. As another example, a clinician may administer antihypertensive drugs, such as beta blockers (e.g., Labetalol) and ACE-inhibitors (e.g., Enalapril) or may undertake intravascular interventions such as coil embolization to treat hemorrhagic stroke. Lastly, if stroke symptoms have resolved on their own with negative neurological work-up, a clinician may administer long-term cardiac monitoring (external or implantable) to determine potential cardiac origins of cryptogenic stroke.

### SUMMARY

Detecting cranial acute health events using observational symptoms and/or electroencephalography (EEG) signal(s) may be confounded by cranial acute health event mimics, which may lead to false-positive detection. A medical device or system configured to detect cranial acute health events that has many false-positive detection results, such as falsely indicating a cranial acute health event mimic as a cranial acute health event, may be considered unreliable, such as by a clinician or patient, which may lead to cranial acute health event detection results being ignored or dismissed and/or the medical device or system not being used to detect cranial acute health events. In general, the disclosure is directed to devices, systems, and techniques for determining an initial indication of a cranial acute health event, via a medical device, e.g., an implantable medical device (IMD) located on a head of a patient, and determining whether the initial indication of a cranial acute health event is a cranial acute health event mimic or a cranial acute health event. In some examples, a cranial acute health event may include a stroke.

While the cranial acute health event detection techniques discussed herein may be directed to detecting a stroke, the devices, systems, and techniques may additionally or alternatively be applied in the same or a similar manner to detect cranial health events such as brain ischemia, and/or hypoxia events. For example, using electrodes, the IMD may sense electrical signals from a patient and generate EEG signal(s) based on the electrical signals.

In some examples, processing circuitry of the system, e.g., of the IMD or another device configured to communicate with the IMD, may determine, based on one or more EEG signals during a first period of time, an initial indication of a cranial acute health event during the first period of time, and determine whether difference(s) between the at least one particular feature of the one or more EEG signals generated during the first period of time and at least one corresponding particular feature of the one or more EEG signals generated during a second period of time satisfy a cranial acute health event mimic threshold. In response to the difference(s) satisfying the cranial acute health event mimic threshold, processing circuitry may determine the initial indication of cranial acute health event during the first period of time is a cranial acute health event mimic. In response to the difference(s) not satisfying the cranial acute health event mimic threshold processing circuitry may verify the initial indication of cranial acute health event during the first period of time is a cranial acute health event.

In some examples, processing circuitry of the system, e.g., of the IMD or another device configured to communicate with the IMD, may determine, based on one or more EEG signals, an initial indication of a cranial acute health event based on at least one particular feature of the one or more EEG signals of a first brain hemisphere. Processing circuitry may determine whether difference(s) between the at least one particular feature of the one or more EEG signals of the first brain hemisphere and at least one corresponding particular feature of the one or more EEG signals of a second brain hemisphere satisfy a cranial acute health event brain hemisphere mimic threshold. In response to the differences satisfying the cranial acute health event brain hemisphere mimic threshold, processing circuitry may determine the initial indication of cranial acute health event is a cranial acute health event mimic. In response to the differences not satisfying the cranial acute health event brain hemisphere mimic threshold, processing circuitry may verify the initial indication of cranial acute health event is a cranial acute health event.

The techniques of this disclosure may provide one or more advantages. For example, the use of difference(s) between the at least one particular feature of the one or more EEG signals of different time periods and/or of different brain hemispheres to determine whether an initial indication of a cranial acute health event is a cranial acute health event mimic or a cranial acute health event may improve cranial acute health event detection by the device or system. For example, the techniques of this disclosure may help generate an indication of cranial acute health event detection with greater specificity and/or sensitivity by selectively filtering out cranial acute health event mimics. In some examples, the techniques of this disclosure to selectively filter out cranial acute health event mimics may improve the specificity and/or sensitivity of detecting cranial acute health event(s) by reducing false indications of cranial acute health event(s) caused by a cranial acute health event mimic being falsely detected as a cranial acute health event. The techniques of this disclosure improving the specificity and/or sensitivity of detecting cranial acute health event medical device or system by selectively filtering out cranial acute health event mimics may lead to the medical device or system, as discussed herein, to be effective and relied upon, such as by a clinician or patient, to reliably detect a cranial acute health event, which improves the usefulness of the device or system described herein.

In one example, this disclosure describes a system comprising a plurality of electrodes; sensing circuitry configured to: sense, via at least two electrodes of the plurality of electrodes, electrical signals from a patient; and generate, based on the electrical signals, one or more EEG signals; and processing circuitry configured to: receive, from the sensing circuitry, one or more EEG signals generated during a first period of time; determine, based on at least one particular feature of the one or more EEG signals generated during the first period of time, an initial indication of a cranial acute health event during the first period of time; receive, from the sensing circuitry, one or more EEG signals generated during a second period of time; determine whether a difference between the at least one particular feature of the one or more EEG signals generated during the first period of time and at least one corresponding particular feature of the one or more EEG signals generated during the second period of time satisfies a cranial acute health event mimic threshold; in response to the difference satisfying the cranial acute health event mimic threshold, determine the initial indication of cranial acute health event during the first period of time is a cranial acute health event mimic; and in response to the difference not satisfying the cranial acute health event mimic threshold, verify the initial indication of cranial acute health event during the first period of time is a cranial acute health event.

In another example, this disclosure describes a system comprising: a plurality of electrodes; sensing circuitry configured to: sense, via at least two electrodes of the plurality of electrodes, electrical signals from a patient; and generate, based on the electrical signals, one or more EEG signals; and processing circuitry configured to: receive, from the sensing circuitry, one or more EEG signals of a first brain hemisphere; determine, based on at least one particular feature of the one or more EEG signals of the first brain hemisphere, an initial indication of a cranial acute health event; receive, from the sensing circuitry, one or more EEG signals of a second brain hemisphere; determine whether a difference between the at least one particular feature of the one or more EEG signals of the first brain hemisphere and at least one corresponding particular feature of the one or more EEG signals of the second brain hemisphere satisfies a cranial acute health event brain hemisphere mimic threshold; in response to the difference satisfying the cranial acute health event brain hemisphere mimic threshold, determine the initial indication of cranial acute health event is a cranial acute health event mimic; and in response to the difference not satisfying the cranial acute health event brain hemisphere mimic threshold, verify the initial indication of cranial acute health event is a cranial acute health event.

In another example, this disclosure describes a method comprising: sensing, by sensing circuitry and via at least two electrodes of a plurality of electrodes, electrical signals from a patient; generating, by the sensing circuitry and based on the electrical signals, one or more EEG signals; receiving, by processing circuitry and from the sensing circuitry, one or more EEG signals generated during a first period of time; determining, by the processing circuitry and based on at least one particular feature of the one or more EEG signals generated during the first period of time, an initial indication of a cranial acute health event during the first period of time; receiving, by the processing circuitry and from the sensing circuitry, one or more EEG signals generated during a second period of time; determining, by the processing circuitry, whether a difference between the at least one particular feature of the one or more EEG signals generated during the first period of time and at least one corresponding particular feature of the one or more EEG signals generated during the second period of time satisfies a cranial acute health event mimic threshold; in response to the difference satisfying the cranial acute health event mimic threshold, determining, by the processing circuitry, the initial indication of cranial acute health event during the first period of time is a cranial acute health event mimic; and in response to the difference not satisfying the cranial acute health event mimic threshold, verifying, by the processing circuitry, the initial indication of cranial acute health event during the first period of time is a cranial acute health event.

In another example, this disclosure describes a method comprising: sensing, by sensing circuitry and via at least two electrodes of a plurality of electrodes, electrical signals from a patient; generating, by the sensing circuitry and based on the electrical signals, one or more EEG signals; receiving, by processing circuitry and from the sensing circuitry, one or more EEG signals of a first brain hemisphere; determining, by the processing circuitry and based on at least one particular feature of the one or more EEG signals of the first brain hemisphere, an initial indication of a cranial acute health event; receiving, by the processing circuitry and from the sensing circuitry, one or more EEG signals of a second brain hemisphere; determining, by the processing circuitry, whether a difference between the at least one particular feature of the one or more EEG signals of the first brain hemisphere and at least one corresponding particular feature of the one or more EEG signals of the second brain hemisphere satisfies a cranial acute health event brain hemisphere mimic threshold; in response to the difference satisfying the cranial acute health event brain hemisphere mimic threshold, determining, by the processing circuitry, the initial indication of cranial acute health event is a cranial acute health event mimic; and in response to the difference not satisfying the cranial acute health event brain hemisphere mimic threshold, verifying, by the processing circuitry, the initial indication of cranial acute health event is a cranial acute health event.

In another example, this disclosure describes a computer-readable medium comprising instructions that, when executed, cause processing circuitry to execute sensing, by sensing circuitry and via at least two electrodes of a plurality of electrodes, electrical signals from a patient; generating, by the sensing circuitry and based on the electrical signals, one or more EEG signals; receiving, by processing circuitry and from the sensing circuitry, one or more EEG signals generated during a first period of time; determining, by the processing circuitry and based on at least one particular feature of the one or more EEG signals generated during the first period of time, an initial indication of a cranial acute health event during the first period of time; receiving, by the processing circuitry and from the sensing circuitry, one or more EEG signals generated during a second period of time; determining, by the processing circuitry, whether a difference between the at least one particular feature of the one or more EEG signals generated during the first period of time and at least one corresponding particular feature of the one or more EEG signals generated during the second period of time satisfies a cranial acute health event mimic threshold; in response to the difference satisfying the cranial acute health event mimic threshold, determining, by the processing circuitry, the initial indication of cranial acute health event during the first period of time is a cranial acute health event mimic; and in response to the difference not satisfying the cranial acute health event mimic threshold, verifying, by the processing circuitry, the initial indication of cranial acute health event during the first period of time is a cranial acute health event.

In another example, this disclosure describes a computer-readable medium comprising instructions that, when executed, cause processing circuitry to execute sensing, by sensing circuitry and via at least two electrodes of a plurality of electrodes, electrical signals from a patient; generating, by the sensing circuitry and based on the electrical signals, one or more EEG signals; receiving, by processing circuitry and from the sensing circuitry, one or more EEG signals of a first brain hemisphere; determining, by the processing circuitry and based on at least one particular feature of the one or more EEG signals of the first brain hemisphere, an initial indication of a cranial acute health event; receiving, by the processing circuitry and from the sensing circuitry, one or more EEG signals of a second brain hemisphere; determining, by the processing circuitry, whether a difference between the at least one particular feature of the one or more EEG signals of the first brain hemisphere and at least one corresponding particular feature of the one or more EEG signals of the second brain hemisphere satisfies a cranial acute health event brain hemisphere mimic threshold; in response to the difference satisfying the cranial acute health event brain hemisphere mimic threshold, determining, by the processing circuitry, the initial indication of cranial acute health event is a cranial acute health event mimic; and in response to the difference not satisfying the cranial acute health event brain hemisphere mimic threshold, verifying, by the processing circuitry, the initial indication of cranial acute health event is a cranial acute health event.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1A is a conceptual diagram of a system configured to determine whether an initial indication of a cranial acute health event is a cranial acute health event mimic in accordance with examples of the present disclosure.

FIG. 1B is a conceptual diagram of a system configured to determine whether an initial indication of a cranial acute health event is a cranial acute health event mimic in accordance with examples of the present disclosure.

FIG. 1C is a conceptual diagram of a system configured to determine whether an initial indication of a cranial acute health event is a cranial acute health event mimic in accordance with examples of the present disclosure.

FIG. 1D is a diagram of the 10-20 map for electroencephalography (EEG) sensor measurements.

FIG. 2A depicts a top view of a sensor device in accordance with embodiments of the present technology.

FIG. 2B depicts a side view of the sensor device shown in FIG. 2A in accordance with the present technology.

FIG. 2C depicts a top view of another embodiment of sensor device in accordance with the present technology.

FIG. 2D depicts a side view of another embodiment of a sensor device in accordance with the present technology.

FIG. 2E depicts a side view of another embodiment of a sensor device in accordance with the present technology.

FIG. 2F depicts a side view of another embodiment of a sensor device in accordance with the present technology.

FIG. 2G depicts an example sensor device that includes electrode extensions in conjunction with a patient, in accordance with examples of the present disclosure.

FIG. 3 depicts another sensor device in accordance with embodiments of the present technology.

FIG. 4 is a block diagram of an example sensor device configured to detect a cranial acute health event.

FIG. 5 is a block diagram of an example external device configured to communicate with the sensor device of FIG. 4.

FIG. 6 is a block diagram illustrating an example system that includes an access point, a network, external computing devices, such as a server, and one or more other computing devices, which may be coupled to the IMD, the external device, and the processing circuitry of FIG. 1 via a network, in accordance with one or more techniques described herein.

FIG. 7 is an example diagram of differences between particular feature(s) of EEG signal(s) that satisfy a cranial acute health mimic threshold or a cranial acute health event brain hemisphere mimic threshold, in accordance with one or more techniques described herein.

FIGS. 8A-8B are flow diagrams of example techniques for determining whether an initial indication of a cranial acute health event of a patient is a cranial acute health event mimic or is a cranial acute health event.

Many aspects of the present disclosure can be better understood with reference to the following drawings. The components in the drawings are not necessarily to scale. Instead, emphasis is placed on clearly illustrating the principles of the present technology.

### DETAILED DESCRIPTION

It can be difficult to determine whether a patient is suffering from a stroke or has suffered from a stroke. Current diagnostic techniques typically involve evaluating a patient for visible symptoms, such as paralysis or numbness of the face, arm, or leg, as well as difficultly walking, speaking, balancing, secing, or understanding (e.g., the B.E.F.A.S.T visible stroke indication of Balance, Eyes, Face, Arm, Speech, Time to call for emergency help). However, these techniques may result in undiagnosed strokes, particularly more minor strokes that leave patients relatively functional upon cursory evaluation. Other diagnostic techniques may include evaluating imaging, such as a computerized tomography (CT) scan or magnetic resonance imagining (MRI), which usually needs to be performed in a medical office or hospital.

Even for relatively minor strokes, it is important to treat the patient as soon as possible because treatment outcomes for stroke patients are highly time-dependent. However, such treatments may be frequently underutilized and/or relatively ineffective due to the failure to timely identify whether a patient is undergoing or has recently undergone a stroke. This is a particular risk with more minor strokes that leave patients relatively functional upon cursory evaluation. However, detecting strokes using observational symptoms and/or EEG signal(s) may be confounded by stroke mimics, which may lead to false-positive stroke detection results. A medical device configured to detect strokes that has many false-positive detection results, such as falsely indicating a stroke mimic as a stroke, may be considered unreliable, such as by a clinician or patient, which may cause a medical device configured to detect strokes that has many false-positive detection results ineffective.

In some examples, EEG signals fall in the range of 0.5-approximately 200 Hertz (Hz). Waveforms may be subdivided into bandwidths known as delta (δ), theta (θ), alpha (α), beta (β), and gamma (γ). For example, a delta (δ) band may be between 0.5 Hz and 4 Hz, a theta (θ) band may be between 4 Hz and 7 Hz, an alpha (α) band may be between 8 Hz and 12 Hz, a beta (β) band may be between 13 Hz and 30 Hz, and a gamma (γ) band may be between 30 Hz to 200 Hz. In some examples, the disclosure describes techniques for detecting a cranial acute health event, such as a stroke, that use bandwidth characteristic, such as an amplitude of a particular bandwidth or a ratio of the energies in two of these bands as a metric, e.g., to determine a change of the value of the amplitude or ratio (or other metric) over a period of time. Example bandwidth ratios that the techniques of this disclosure may use to detect a cranial acute health event include a delta-alpha ratio (DAR), delta-theta ratio (DTR), a (delta+theta)/(alpha+beta) ratio (DTABR), a beta-alpha ratio (BAR), a gamma-alpha ratio (GAR), and a burst-suppression ratio (BSR). In some examples, the respective ratios may be signal power ratios between the respective frequency bandwidths. In some examples, a BSR may be a fraction of an EEG signal spent in a suppressed state (e.g., an amplitude of EEG signal being below a suppressed state threshold, such as less than 5 micro volts) over a period of time.

Accordingly, there is a need for improved methods for detecting a cranial acute health event, such as strokes, that filters out a greater amount of cranial acute health event mimics to improve the specificity and sensitivity of determining indications of cranial acute health event(s). In general, the disclosure is directed to devices, systems, and techniques for detecting an initial indication of a cranial acute health event, via a medical device, e.g., an IMD or external medical device, located on the head of a patient, and determining whether the initial indication of a cranial acute health event is a cranial acute health event mimic.

This disclosure describes various devices, systems, and techniques for detecting an initial indication of a cranial acute health event, via a medical device, e.g., an implantable medical device (IMD) or external medical device, located on the head of a patient, and determining whether the initial indication of a cranial acute health event is a cranial acute health event mimic.

As described herein, a medical device (e.g., an IMD or external medical device wearable by the patient), may be configured to detect an initial indication of a cranial acute health event, such as stroke, from a location on or near the head of the patient. Using electrodes, the IMD may sense electrical signals from a patient and generate an EEG signal based on the electrical signals. Processing circuitry of the system, such as processing circuitry of an IMD or of another device configured to communicate with the IMD, may determine at least one particular feature of the one or more EEG signals, such as of a particular period of time and/or of a particular brain hemisphere. In some examples, the at least one particular feature comprises a bandwidth characteristic of the one or more EEG signals. In some examples, the bandwidth characteristic includes a bandwidth ratio. In some examples, the bandwidth ratio includes a DAR. In some examples, a bandwidth characteristic may include one or more of a bandwidth ratio or an amplitude of a particular bandwidth of one or more EEG signals. In some examples, a bandwidth ratio may be a ratio of bandwidth of the waveforms of the EEG signals, such as delta (δ), theta (θ), alpha (α), beta (β), and gamma (γ). In some examples, an amplitude bandwidth may be amplitude of a particular waveform of the EEG signals, such as delta (δ), theta (θ), alpha (α), beta (β), and gamma (γ).

In some examples, this disclosure describes techniques for determining an initial indication of a cranial acute health event during a first period of time based on at least one particular feature of the one or more EEG signals generated during the first period of time, determining whether difference(s) between the at least one particular feature of the one or more EEG signals generated during the first period of time and at least one corresponding particular feature of the one or more EEG signals generated during the second period of time satisfy a cranial acute health event mimic threshold, in response to the difference(s) satisfying a cranial acute health event mimic threshold, determining the initial indication of cranial acute health event during the first period of time is a cranial acute health event mimic, and in response to the difference(s) not satisfying the cranial acute health event mimic threshold, verifying the initial indication of cranial acute health event during the first period of time is a cranial acute health event. In some examples, the cranial acute health event includes a stroke.

In some examples, this disclosure describes techniques for determining an initial indication of a cranial acute health event during a first period of time based on at least one particular feature of the one or more EEG signals generated during the first period of time, determining whether difference(s) between the at least one particular feature of the one or more EEG signals generated during the first period of time and at least one corresponding particular feature of the one or more EEG signals generated during a second period of time satisfy a cranial acute health event mimic threshold, in response to the difference(s) satisfying the cranial acute health event mimic threshold, determining the initial indication of cranial acute health event during the first period of time is a cranial acute health event mimic, and in response to the difference(s) not satisfying the cranial acute health event mimic threshold, verifying the initial indication of cranial acute health event during the first period of time is a cranial acute health event. In some examples, the cranial acute health event includes a stroke.

In some examples, in response to either the initial indication of a cranial acute health event, the determination that the initial indication of a cranial acute health event is a cranial acute health event mimic, or the determination that the initial indication of a cranial acute health event is a cranial acute health event the IMD may send patient data, such as one or more of the EEG signal(s), to another computing device, such as a patient's smartphone and/or a server, for further adjudication. In some examples, the further adjudication may include the another computing device confirming or denying whether the cranial acute health event was detected by the IMD and/or confirming or denying what type of acute health event was detected. In some examples, the further adjudication may include the another computing device applying an artificial intelligence model (e.g., machine learning, neural networks, etc.) to patient acute health event data to confirm or deny the cranial acute health event detected by the IMD and/or confirm or deny what type of cranial acute health event was detected.

Conventional EEG electrodes are typically positioned over a large portion of a user's scalp. While electrodes in this region are well positioned to detect electrical activity from the patient's brain, there are certain drawbacks. Sensors in this location interfere with patient movement and daily activities, making them impractical for prolonged monitoring. Additionally, implanting traditional electrodes under the patient's scalp is difficult and may lead to significant patient discomfort. To address these and other shortcomings of conventional EEG sensors, embodiments of the present technology include an IMD configured to record electrical signals at a region near the patient's head, such as adjacent a rear portion of the patient's neck or base the patient's skull or near the patient's temple. In these positions, implantation under the patient's skin is relatively simple, and a temporary application of a wearable sensor device (e.g., coupled to a bandage, garment, band, or adhesive member) does not unduly interfere with patient movement and activity. Although primarily described in the context of leadless sensor devices, in some examples, a sensor device may include electrode extensions. The electrode extensions may increase a size of a vector for sensing signals via the electrodes, such as brain and cardiac signals, and/or may position electrodes closer to a source of the brain and cardiac signals, which may enhance the sensitivity of algorithms using such signals to detect and/or predict patient conditions.

However, the EEG signals detected via electrodes disposed at or adjacent the back of a patient's neck may include relatively high noise amplitude. For example, the electrical signals associated with brain activity may be intermixed with electrical signals associated with cardiac activity (e.g., ECG signals) or signals including components associated with mechanical activity of the heart and skeletal muscle activity (e.g., EMG signals) and artifacts from other electrical sources such as patient movement or external interference. Accordingly, in some embodiments, the sensor data may be filtered or otherwise manipulated to separate the brain activity data (e.g., EEG signals) and ECG signals (or other cardiac signals) from each other and other electrical signals (e.g., EMG signals, etc.). In some examples, IMD or an external device may employ machine learning/adaptive neural network techniques to improve the signal extraction capability (e.g., to filter out or reduce the contribution of ECG signals from the EEG signals). One such methodology is described in “ECG Artifact Removal of EEG signal using Adaptive Neural Network” as published in IEEE Xplore 27 May 2019, which is hereby incorporated by reference in its entirety. Similarly, electrical signals associated with skeletal muscle activity may also be filtered from the EEG sensor data to remove such artifacts.

Aspects of the technology described herein can be embodied in a special purpose computer or data processor that is specifically programmed, configured, or constructed to perform one or more of the computer-executable instructions explained in detail herein. Aspects of the technology can also be practiced in distributed computing environments where tasks or modules are performed by remote processing devices, which are linked through a communication network (e.g., a wireless communication network, a wired communication network, a cellular communication network, the Internet, a short-range radio network (e.g., such as via Bluetooth)). In a distributed computing environment, program modules may be located in both local and remote memory storage devices.

Computer-implemented instructions, data structures, screen displays, and other data under aspects of the technology may be stored or distributed on computer-readable storage media, including magnetically or optically readable computer disks, as microcode on semiconductor memory, nanotechnology memory, organic or optical memory, or other portable and/or non-transitory data storage media. In some embodiments, aspects of the technology may be distributed over the Internet or over other networks (e.g., a Bluetooth network) on a propagated signal on a propagation medium (e.g., an electromagnetic wave(s), a sound wave) over a period of time, or may be provided on any analog or digital network (packet switched, circuit switched, or other scheme).

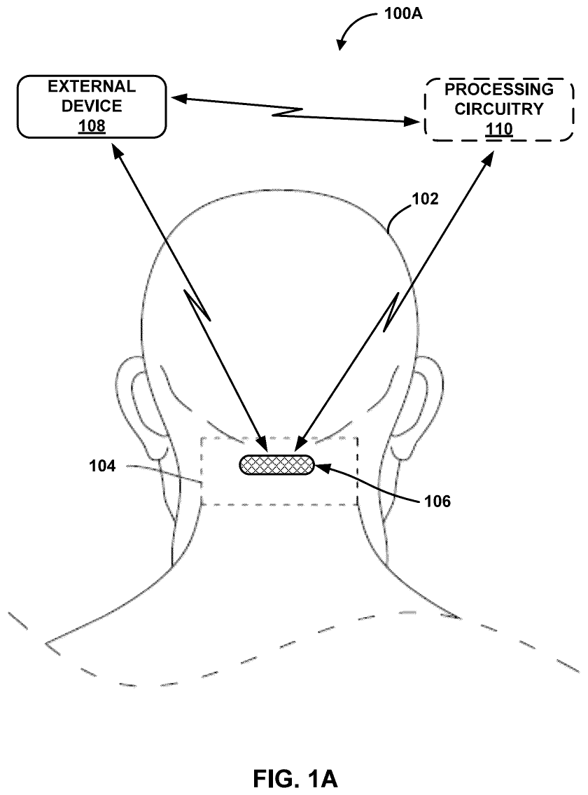

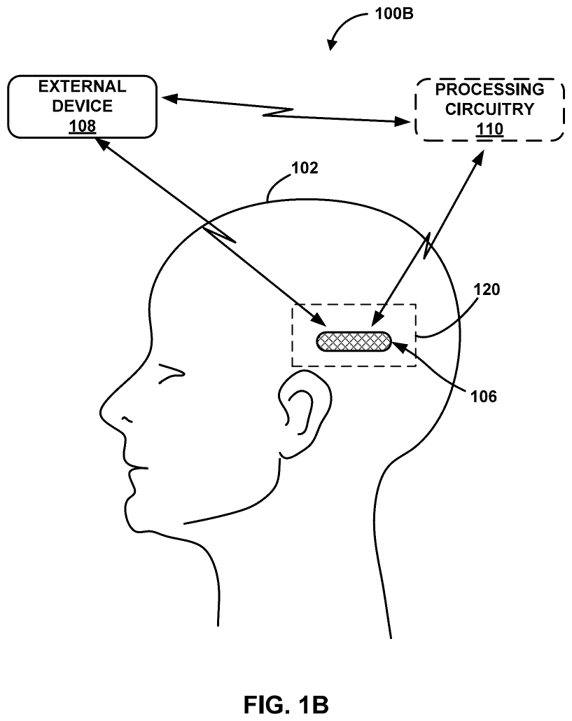

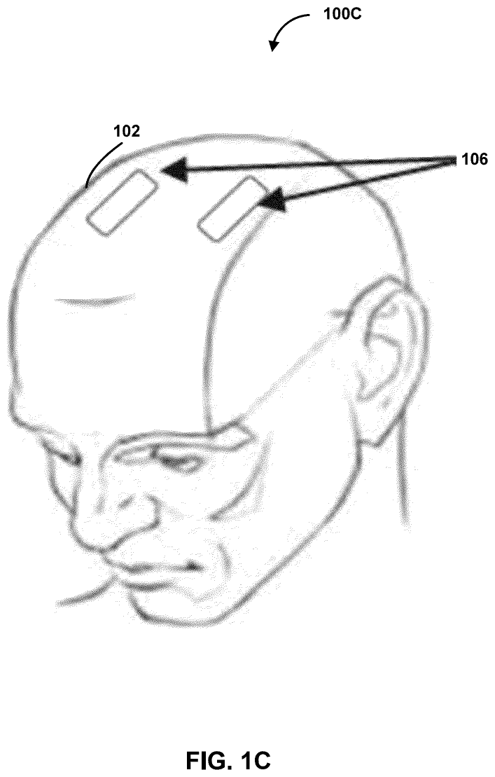

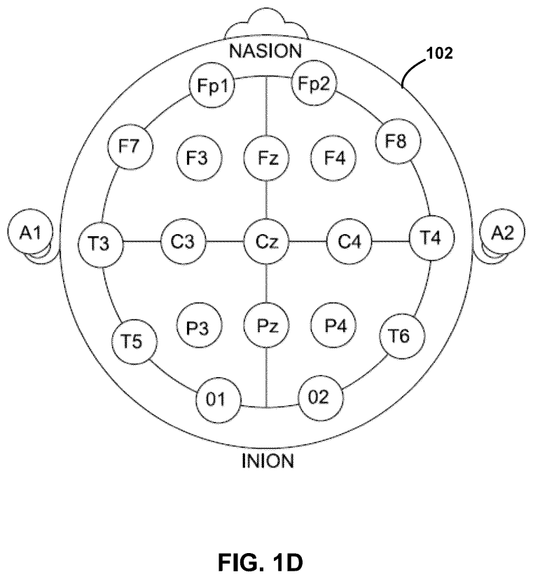

FIG. 1A is a conceptual diagram of a system 100A configured to determine an initial indication of a cranial acute health event and determine whether the initial indication of a cranial acute heath event is a cranial acute health event mimic in accordance with examples of the present disclosure. The example techniques described herein may be used with an implantable medical device (IMD) 106, which may be in wireless communication with at least one of external device 108, processing circuitry 110, and other devices not pictured in FIG. 1A. For example, an external device (not illustrated in FIG. 1A) may include at least a portion of processing circuitry 110, the external device configured for communication with IMD 106, and external device 108. As shown in FIG. 1A, IMD 106 is located in target region 104. Target region 104 can be a rear portion of a user's neck or at the base of the skull. Although IMD 106 may be implanted at a location generally centered with respect to the head, neck, or target region 104, IMD 106 may be implanted in an off-center location in order to obtain desired vectors from the electrodes carried on the housing of IMD 106. In other examples, target region may be located at other positions of patient, such as near the user's temple(s) (e.g., above the car(s)) and/or over the temporal portion of the skull. IMD 106 can be disposed in target region 104 either via implantation (e.g., subcutaneously) or by being placed over the patient's skin with one or more electrodes of IMD 106 being in direct contact with the patient's skin at or adjacent the target region 104. In some examples, e.g., as shown in FIG. 1C, the system may include plurality of IMDs 106, such as two or more IMDs 106 configured to individually and/or cooperatively detect a cranial acute health event in accordance with examples of the present disclosure.

While conventional EEG electrodes are placed over the patient's scalp, the present technology advantageously enables recording of clinically useful brain activity data via electrodes positioned at the target region 104 at the rear of the patient's neck or head, or other cranial locations, such as temporal locations, described herein. This anatomical area is well suited to both implantation of IMD 106 and to temporary placement of a sensor device over the patient's skin. In contrast, conventional EEG electrodes positioned over the scalp are cumbersome, and implantation over the patient's skull is challenging and may introduce significant patient discomfort. As noted elsewhere here, conventional EEG electrodes are typically positioned over the scalp to more readily achieve a suitable signal-to-noise ratio for detection of brain activity. However, by using certain digital signal processing, and a special-purpose classifier algorithm, clinically useful brain activity data can be obtained using sensors disposed at the target region 104. Specifically, the electrodes can detect electrical activity that corresponds to brain activity in the P3, Pz, and/or P4 regions (as shown in FIG. 1D).

While conventional approaches to stroke detection utilizing EEG have relied on data from a large number of EEG electrodes, this disclosure describes that clinically useful cranial acute health event determinations can be made utilizing relatively few electrodes, such as via the electrodes carried by IMD 106. For example, IMD 106 may extract features from EEG signals indicative of brain activity or cardiac activity. IMD 106 may then determine whether or not the patient has experienced a cranial acute health event based on these extracted features. In some examples, IMD 106 takes the form of a LINQ™ Insertable Cardiac Monitor (ICM), available from Medtronic, Inc., of Minneapolis, Minnesota. The example techniques may additionally, or alternatively, be used with a medical device not illustrated in FIG. 1A such as another type of IMD, a patch monitor device, a wearable device (e.g., smart watch), or another type of external medical device.

Clinicians sometimes diagnose a patient (e.g., patient 102) with medical conditions and/or determine whether a condition of patient 102 is improving or worsening based on one or more observed physiological signals collected by physiological sensors, such as electrodes, optical sensors, chemical sensors, temperature sensors, acoustic sensors, and motion sensors. In some cases, clinicians apply non-invasive sensors to patients in order to sense one or more physiological signals while a patient is in a clinic for a medical appointment. However, in some examples, events that may change a condition of a patient, such as administration of a therapy, may occur outside of the clinic. As such, in these examples, a clinician may be unable to observe the physiological markers needed to determine whether a cranial acute health event, such as a stroke, has changed a medical condition of the patient and/or determine whether a medical condition of the patient is improving or worsening while monitoring one or more physiological signals of the patient during a medical appointment. In the example illustrated in FIG. 1A, IMD 106 is implanted within patient 102 to continuously record one or more physiological signals of patient 102 over an extended period of time.

In some examples, IMD 106 includes a plurality of electrodes. The plurality of electrodes is configured to detect signals that enable processing circuitry of IMD 106 to determine current values of cranial acute health event metrics associated with the brain and/or cardiovascular functions of patient 102. In some examples, the plurality of electrodes of IMD 106 are configured to detect a signal indicative of an electric potential of the tissue surrounding the IMD 106. Moreover, IMD 106 may additionally or alternatively include one or more optical sensors, accelerometers, impedance sensors, respiration sensors, temperature sensors, chemical sensors, light sensors, pressure sensors, and acoustic sensors, in some examples. Such sensors may detect one or more physiological parameters indicative of a patient condition.

External device 108 may be a hand-held computing device with a display viewable by the user and an interface for providing input to external device 108 (e.g., a user input mechanism). In some examples, external device 108 may be a smartphone, smart watch, smart glasses, or other personal smart device. In some examples, external device 108 may be a smart device of patient 102. For example, external device 108 may include a small display screen (e.g., a liquid crystal display (LCD) or a light emitting diode (LED) display) that presents information to the user. In addition, external device 108 may include a touch screen display, keypad, buttons, a peripheral pointing device, voice activation, or another input mechanism that allows the user to navigate through the user interface of external device 108 and provide input. If external device 108 includes buttons and a keypad, the buttons may be dedicated to performing a certain function, e.g., a power button, the buttons and the keypad may be soft keys that change in function depending upon the section of the user interface currently viewed by the user, or any combination thereof.

In other examples, external device 108 may be a larger workstation or a separate application within another multi-function device, rather than a dedicated computing device. For example, the multi-function device may be a notebook computer, tablet computer, workstation, one or more servers, cellular phone, personal digital assistant, or another computing device that may run an application that enables the computing device to operate as a secure device.

When external device 108 is configured for use by the clinician, external device 108 may be used to transmit instructions to IMD 106. Example instructions may include requests to set electrode combinations for sensing and any other information that may be useful for programming into IMD 106. To program IMD 106, the clinician may configure and store operational parameters for IMD 106 within IMD 106 with the aid of external device 108. In some examples, external device 108 assists the clinician in the configuration of IMD 106 by providing a system for identifying potentially beneficial operational parameter values.

Whether external device 108 is configured for clinician or patient use, external device 108 is configured to communicate with IMD 106 and, optionally, another computing device (not illustrated by FIG. 1A), via wireless communication. External device 108, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies). In some examples, external device 108 is a smartphone of patient 102 and/or a watch or other wearable computing device, which may communicate with IMD 106, e.g., via Bluetooth™. In some examples, external device 108 is configured to communicate with a computer network, such as the Medtronic CareLink® Network developed by Medtronic, plc, of Dublin, Ireland. For example, external device 108 may send data, such as data received from IMD 106, to another external device such as a smartphone, a tablet, or a desktop computer, and the other external device may in turn send the data to the computer network. In other examples, external device 108 may directly communicate with the computer network without an intermediary device.

Processing circuitry 110, in some examples, may include one or more processors that are configured to implement functionality and/or process instructions for execution within IMD 106. For example, processing circuitry 110 may be capable of processing instructions stored in a storage device. Processing circuitry 110 may include, for example, microprocessors, graphical processing units (GPUs), tensor processing units (TPUs), digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 110 may include any suitable structure, whether in hardware, software, firmware, or any combination thereof, to perform the functions ascribed herein to processing circuitry 110.

Processing circuitry 110 may represent processing circuitry located within any one or both of IMD 106 and external device 108. In some examples, processing circuitry 110 may be entirely located within a housing of IMD 106. In other examples, processing circuitry 110 may be entirely located within a housing of external device 108. In other examples, processing circuitry 110 may be located within any one or combination of IMD 106, external device 108, and another device or group of devices that are not illustrated in FIG. 1A. As such, techniques and capabilities attributed herein to processing circuitry 110 may be attributed to any combination of IMD 106, external device 108, and other devices that are not illustrated in FIG. 1A.

Medical device system 100A of FIG. 1A is an example of a system configured to collect electrical signals and generate cranial acute health event metrics, such as stroke metrics, according to one or more techniques of this disclosure. In some examples, processing circuitry 110 includes sensing circuitry configured to generate physiological information from the sensed electrical signal of patient 102. In one example, an electrical signal is sensed via one or more electrode combinations of IMD 106. An electrical signal is representative of electrical activity of the brain, heart, or other physiological functions as measured by electrodes implanted within the body. The sensed electrical signals may include features representative of brain function, such as amplitudes of frequencies in one or more frequency bands, such as alpha bands, beta bands, delta bands, or gamma bands. Brain signal analysis circuitry, which may be implemented as part of processing circuitry 110 may perform various processing circuitry to extract these brain features from the sensed electrical signals. In some examples, the sensed electrical signals may include features representative of heart function, such as P-waves (depolarization of the atria), R-waves (depolarization of the ventricles), and T-waves (repolarization of the ventricles), among other events.

In some examples, IMD 106 includes one or more accelerometers. An accelerometer of IMD 106 may collect an accelerometer signal which reflects a measurement of any one or more of a motion of patient 102, a posture of patient 102 and a body angle of patient 102. In some cases, the accelerometer may collect a three-axis accelerometer signal indicative of patient 102's movements within a three-dimensional Cartesian space. For example, the accelerometer signal may include a vertical axis accelerometer signal vector, a lateral axis accelerometer signal vector, and a frontal axis accelerometer signal vector. The vertical axis accelerometer signal vector may represent an acceleration of patient 102 along a vertical axis, the lateral axis accelerometer signal vector may represent an acceleration of patient 102 along a lateral axis, and the frontal axis accelerometer signal vector may represent an acceleration of patient 102 along a frontal axis. In some cases, the vertical axis substantially extends along a torso of patient 102 when patient 102 from a neck of patient 102 to a waist of patient 102, the lateral axis extends across a chest of patient 102 perpendicular to the vertical axis, and the frontal axis extends outward from and through the chest of patient 102, the frontal axis being perpendicular to the vertical axis and the lateral axis.

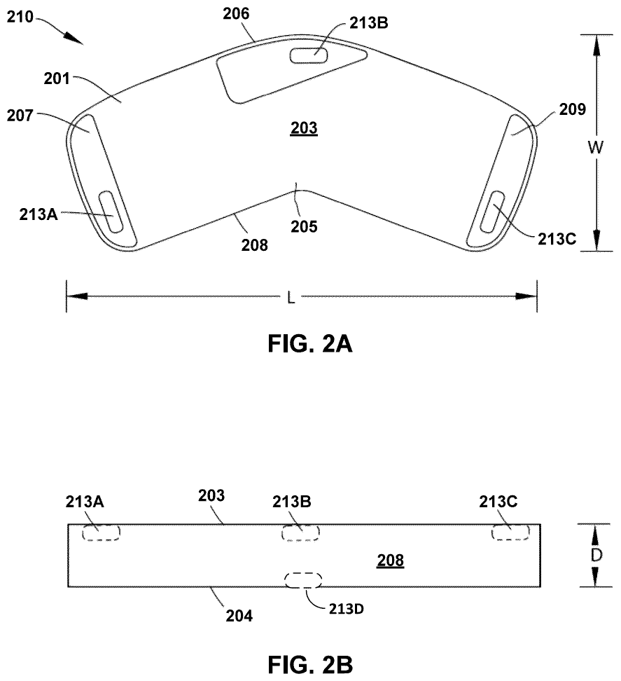

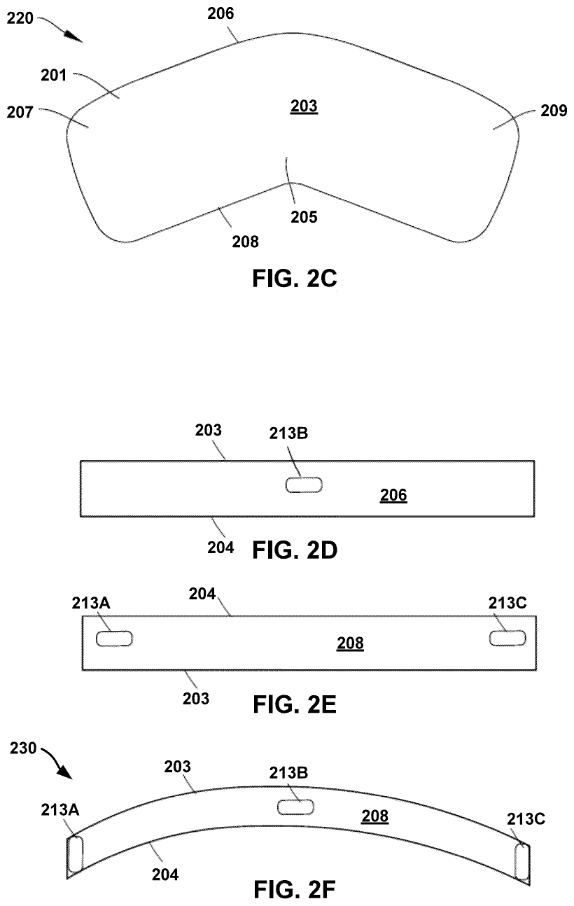

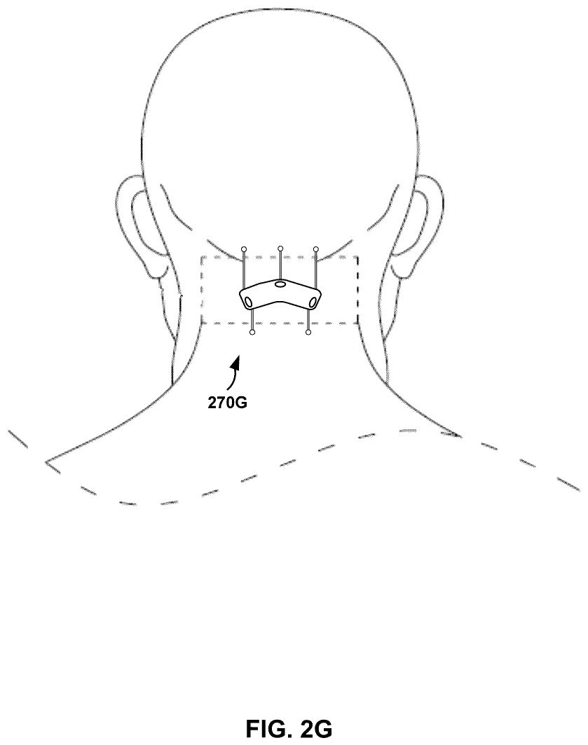

IMD 106 may additionally or alternatively measure a set of parameters including an impedance (e.g., subcutaneous impedance measured via electrodes depicted in FIGS. 2A-2G, an intrathoracic impedance or an intracardiac impedance) of patient 102, a respiratory rate of patient 102 during night hours, a respiratory rate of patient 102 during day hours, a heart rate of patient 102 during night hours, a heart rate of patient 102 during day hours, an atrial fibrillation (AF) burden of patient 102, a ventricular rate of patient 102 while patient 102 is experiencing AF, or any combination thereof. Processing circuitry 110 may analyze any one or more of the set of parameters in order to determine whether or not the patient is experiencing stroke, and may indicate an efficacy of a treatment program administered to patient 102. In some examples, pulsatile signals sensed optically or mechanically, e.g., via the electrodes, an optical sensor, accelerometer, pressure sensor, impedance sensor, or heart sound sensor, from the scalp vasculature may provide a surrogate for an ECG or other cardiac electrical activity signal. In some examples, the treatment program may include treatment delivered by one or more medical devices such as ICDs with intravascular or extravascular leads, pacemakers, CRT-Ds, neuromodulation devices, LVADs, implantable sensors, orthopedic devices, or drug pumps. Additionally, or alternatively, the treatment program may include in-clinic treatments administered by medical professionals, prescribed pharmaceutical regimens, treatments administered by one or more external medical devices, or any combination thereof. In any case, processing circuitry 110 may determine the efficacy of the treatment program by determining a time in which the treatment program is administered (e.g., including a time in which the treatment program begins and/or a time in which the treatment program ends) and analyzing values of any one or combination of the set of parameters relative to the time in which the treatment program is administered. Alternatively, in some examples, processing circuitry 110 may determine the efficacy of a treatment program by evaluating one or more parameters on a rolling basis in order to determine whether the one or more parameters have changed over a period of time.

In some examples, one or more sensors (e.g., electrodes, motion sensors, optical sensors, temperature sensors, or any combination thereof) of IMD 106 may generate a signal that indicates a parameter of a patient. In some examples, the signal that indicates the parameter includes a plurality of parameter values, where each parameter value of the plurality of parameter values represents a measurement of the parameter at a respective interval of time. The plurality of parameter values may represent a sequence of parameter values, where each parameter value of the sequence of parameter values are collected by IMD 106 at a start of each time interval of a sequence of time intervals. For example, IMD 106 may perform a parameter measurement in order to determine a parameter value of the sequence of parameter values according to a recurring time interval (e.g., every day, every night, every other day, every twelve hours, every hour, or any other recurring time interval). In this way, IMD 106 may be configured to track a respective patient parameter more effectively as compared with a technique in which a patient parameter is tracked during patient visits to a clinic, since IMD 106 is implanted within patient 102 and is configured to perform parameter measurements according to recurring time intervals without missing a time interval or performing a parameter measurement off schedule. Processing circuitry 110 may determine these different parameters separately from the cranial acute health event metrics or determine the cranial acute health event metrics based at least partially on one or more other parameter measurements.

IMD 106 may be referred to as a system or device. In one example, IMD 106 may include a memory, a plurality of electrodes carried by the housing of IMD 106, sensing circuitry configured to sense, via at least two electrodes of the plurality of electrodes, electrical signals from patient 102 and generate, based on the electrical signals, physiological information. IMD 106 may also include processing circuitry configured to receive, from the sensing circuitry, the physiological information and determine, based on the physiological information, a cranial acute health event metric indicative of a cranial acute health event status of the patient. The processing circuitry may be configured to then store the cranial acute health event metric in the memory. The housing of IMD 106 carries the plurality of electrodes and contains, or houses, both of the sensing circuitry and the processing circuitry. In this manner, IMD 106 may be referred to as a leadless sensing device because the electrodes are carried directly by the housing instead of by any leads that extend from the housing. In some examples, however, IMD 106 may include one or more sensing leads extending therefrom and into the tissue of the patient; such lead(s) may be employed instead of or in addition to the electrodes of IMD 106, and may perform any of the functions attributed herein to the electrodes.

The physiological data can include electrical brain activity data and/or electrical heart activity data. In some examples, the plurality of electrodes are configured to detect brain activity data corresponding to activity in at least one of a P3, Pz, or P4 brain region, which is at the back of the head or upper neck region as shown in FIG. 1D. In this manner, the housing of IMD 106 may be configured to be disposed at or adjacent to a rear portion of a neck or skull of patient 102. The housing of IMD 106 may be configured to be implanted within patient 102, such as implanted subcutaneously. In other examples, the housing of IMD 106 may be configured to be disposed on an external surface of skin of patient 102.

In some examples, IMD 106 may include a single sensing circuitry configured to generate, from the sensed electrical signals, information that includes both the electrical brain activity data (e.g., EEG data) and the electrical heart activity data (e.g., ECG data or cardiac contraction). In other examples, the processing circuitry of IMD 106 may include separate hardware that generates different information from the sensed electrical signals. For example, IMD 106 may include first circuitry configured to generate the electrical brain activity from the electrical signals and second circuitry different from the first circuitry and configured to generate the electrical heart activity data from the electrical signals. Even with the first and second circuitry configured to generate different information, or data, in some examples, sensed electrical signals may be conditioned or processed by one or more electrical components (e.g., filters or amplifiers) prior to being processed by the first and second circuitry. In some examples, electrical brain activity data may include features, such as spectral features, indicative of the strength of signals in various frequency bands or at various frequencies. In some examples, electrical heart activity data may include features such as the timing and/or amplitude of P-waves, R-waves, or any other features representative of heart function.

Each of the cranial acute health event metrics may be indicative of the likelihood (or risk) that patient 102 has experienced, or is experiencing, a cranial acute health event, respectively. For example, each cranial acute health event metric may include a numerical value representative of the probability that patient 102 has experienced a cranial acute health event. IMD 106 may then compare the metric to a respective threshold or monitor a relative change in the metric value over time to determine whether or not a cranial acute health event occurred or is occurring. In other examples, the cranial acute health event may be a binary value that indicates no event occurred or that an event did occur. In some examples, IMD 106 may generate each cranial acute health event metric based on sensed data other than the sensed electrical signals from the carried electrodes on the housing of IMD 106.

In one example, IMD 106 may include one or more accelerometers within the housing. The accelerometer may be configured to generate accelerometer signal, which may be stored processed as motion/posture data, representative of posture and/or motion of patient 102. IMD 106 may then be configured to determine one or more of a posture of a patient or activity level of a patient based on the generated accelerometer signal.

In some examples, the physiological information generated from the sensed electrical signals may include ECG information. IMD 106 may extract various features from the ECG information, such as heart rate, heart rate variability, etc.

IMD 106 may generate the cranial acute health event metrics at the same or different frequencies. For example, for a patient who has suffered a cranial acute health event in the recent past, such as the past three months, IMD 106 may generate cranial acute health event metrics hourly or daily. In some examples, for a patient who has not suffered a cranial acute health event, IMD 106 may generate cranial acute health event metrics at longer intervals, such as daily or weekly. These time periods are examples, and the generation of cranial acute health event metrics are not limited to the periods discussed above. In some examples, these frequencies may refer to the frequency at which the sensing circuitry generates appropriate information from which the cranial acute health event metric is determined. In other examples, IMD 106 may continually generate physiological information from which cranial acute health event metrics can be determined. However, the frequency may refer to how often the processing circuitry generates the cranial acute health event metric from the physiological information. Continually generating physiological information may include sensing physiological signal and other generation of physiological information on a periodic and/or triggered basis without user intervention.

FIG. 1B is a conceptual diagram of a system 100B configured to determine an initial indication of a cranial acute health event and determine whether the initial indication of a cranial acute heath event is a cranial acute health event mimic in accordance with examples of the present disclosure. System 100B may be substantially similar to system 100A of FIG. 1A. However, system 100B may be configured to be implanted in target region 120 which is located on the side of the head posterior of the temple of patient 102, e.g., above the car and/or over the temporal portion of the cranium. IMD 106 implanted at target region 120 may be configured to generate cranial acute health event metrics based on electrical signals sensed in this area. In such examples, the electrodes of IMD 106 may detect electrical activity that corresponds to brain activity in the T3 region (as shown in FIG. 1D), or T4 region if implanted on the other side of the patient's head, or both of two or more sensor devices are implanted bilaterally at temporal regions. In some examples, IMD 106 may need to employ different filters or other processing or signal conditioning techniques than those at target region 104 due to different types of noise at target region 120, such as muscle activity due to mandible movement or other types of electrical activity.

FIG. 1C is a conceptual diagram of a system 100C configured to determine an initial indication of a cranial acute health event and determine whether the initial indication of a cranial acute heath event is a cranial acute health event mimic in accordance with examples of the present disclosure. System 100C may be substantially similar to system 100A of FIG. 1A or system 100B of FIG. 1B. However, system 100C may be configured to include a plurality of IMDs 106, such as two or more IMDs 106, to be located on the head of patient 102.

Each of IMDs 106 may include a respective set of electrodes and be configured to sense respective EEG signals via the respective electrode (and/or other physiological parameters via other sensors or the electrodes as described herein). In the example, illustrated in FIG. 1C, IMDs 106 (and consequently their respective electrodes) are positioned to detect EEG signals of respective areas, e.g., hemispheres, of the brain of patient 102. Systems (e.g., processing circuitry) described herein may use different localized EEG signals to localize the cranial acute health event, such as stroke, e.g., to a particular hemisphere, or for other purposes related to diagnosing such events as described herein. In some examples, e.g., as described with respect to FIG. 2G, a single IMD may include electrodes coupled thereto via extensions, which may be positioned in different hemispheres or other regions to similar acquire localized EEG signals.

FIG. 1D is a diagram of the 10-20 map for EEG sensor measurements. As shown in FIG. 1D, various locations on the head of patient 102 may be targeted using the electrodes carried by IMD 106. In some examples, the various locations may include one or more of A1, A2, Fp1, Fp2, F7, F3, F2, F4, F8, T3, C3, Cz, C4, T4, T5, P3, Pz, P6, T6, O1, and/or O2. At the back of the head, such as in target region 104 of FIG. 1A, IMD 106 may sense electrical signals at least one of P3, Pz or P4. At the side of the head, such as in target region 120 of FIG. 1B, IMD 106 may sense electrical signals at least one of F7, T3, or T5 adjacent the left hemisphere of the brain. In addition or alternatively, IMD 106 may sense electrical signals at least one of F8, T4, or T6 adjacent the right hemisphere of the brain.

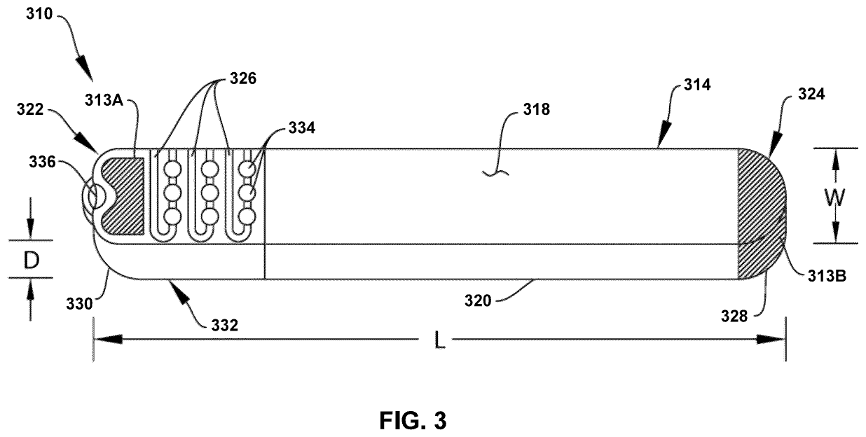

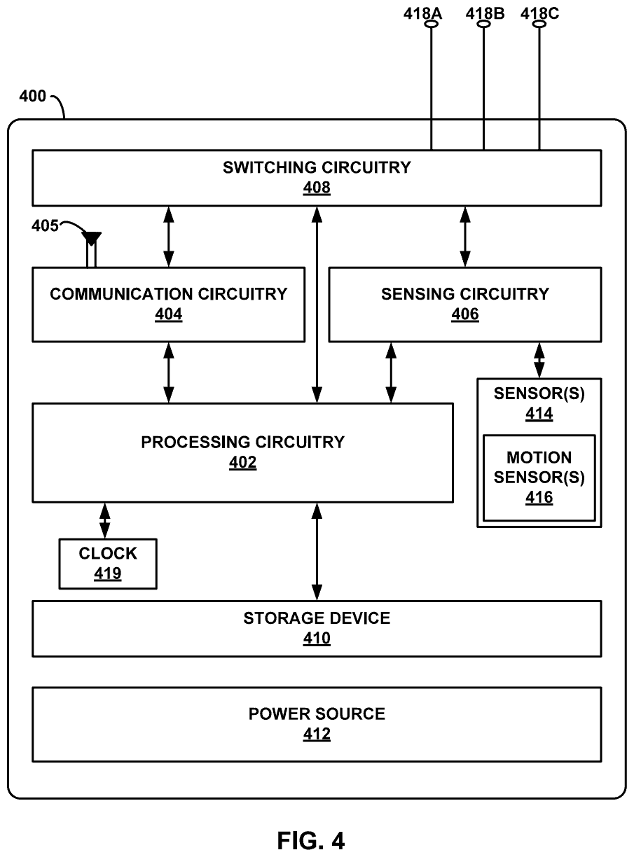

FIG. 2A depicts a top view of a sensor device (e.g., an implantable medical device) in accordance with embodiments of the present technology. FIG. 2B depicts a side view of sensor device 210 shown in FIG. 2A in accordance with the present technology. FIG. 2A illustrates a plane view of an example sensor device 210. In some embodiments, the sensor device 210 can include some or all of the features of, and be similar to, IMD 106 described above with respect to FIGS. 1A and 1B and/or sensor device 310 or IMD 400 described below with respect to FIGS. 3-4, and can include additional features as described in connection with FIG. 2A. In the illustrated example, the sensor device 210 includes a housing 201 that carries a plurality of electrodes 213A, 213B, 213C, 213D (collectively “electrodes 213”) therein. Although four electrodes are shown for sensor device 210, in other examples, only two or three electrodes may be carried or more than four electrodes may be carried by housing 201. In some examples, each electrode may be configured to sense both ECG or other cardiac signals and EEG signals. In other examples, each electrode may be wired to only detect a single type of signal, such as ECG or EEG. In this manner, sensing circuitry may utilize distinct electrodes for sensing EEG and ECG. In such a configuration, two different and independently wired electrodes may be located at the location of each of electrodes 213 shown in FIG. 2A. In operation, electrodes 213 can be placed in direct contact with tissue at the target site (e.g., with the user's skin if placed over the user's skin, or with subcutaneous tissue if the sensor device 210 is implanted). Housing 201 additionally encloses electronic circuitry located inside the sensor device 210 and protects the circuitry (e.g., processing circuitry, sensing circuitry, communication circuitry, sensors, and a power source) contained therein from body fluids. In various embodiments, electrodes 213 can be disposed along any surface of the sensor device 210 (e.g., anterior surface, posterior surface, left lateral surface, right lateral surface, superior side surface, inferior side surface, or otherwise), and the surface in turn may take any suitable form.

In the example of FIGS. 2A and 2B, housing 201 can be a biocompatible material having a relatively planar shape including a first major surface 203 configured to face towards the tissue of interest (e.g., to face anteriorly when positioned at the back of the patient's neck) a second major surface 204 opposite the first, and a depth D or thickness of housing 201 extending between the first and second major surfaces. Housing 201 can define a superior side surface 206 (e.g., configured to face superiorly when sensing device 210 is implanted in or at the patient's head or neck) and an opposing inferior side surface 208. Housing 201 can further include a central portion 205, a first lateral portion (or left portion) 207, and a second lateral portion (or right portion) 209. Electrodes 213 are distributed about housing 201 such that a central electrode 213B is disposed within the central portion 205 (e.g., substantially centrally along a horizontal axis of the device), a back electrode 213D is disposed on inferior side surface, a left electrode 213A electrode is disposed within the left portion 207, and a right electrode 213C is disposed within the right portion 209. As illustrated, housing 201 can define a boomerang or chevron-like shape in which the central portion 205 includes a vertex, with the first and second lateral portions 207 and 209 extending both laterally outward and from the central portion 205 and also at a downward angle with respect to a horizontal axis of the device. In other examples, housing 201 may be formed in other shapes which may be determined by desired distances or angles between different electrodes 213 carried by housing 201.

The configuration of housing 201 can facilitate placement either over the user's skin in a bandage-like form or for subcutaneous implantation. As such, a relatively thin housing 201 can be advantageous. Additionally, housing 201 can be flexible in some embodiments, so that housing 201 can at least partially bend to correspond to the anatomy of the patient's neck (e.g., with left and right lateral portions 207 and 209 of housing 201 bending anteriorly relative to the central portion 205 of housing 201).

In some examples, housing 201 can have a length L of from about 15 to about 50 mm, from about 20 to about 30 mm, or about 25 mm. Housing 201 can have a width W from about 2.5 to about 15 mm, from about 5 to about 10 mm, or about 7.5 mm. In some embodiments, housing 201 can have a thickness of the thickness is less than about 10 mm, about 9 mm, about 8 mm, about 7 mm, about 6 mm, about 5 mm, about 4 mm, or about 3 mm. In some embodiments, the thickness of housing 201 can be from about 2 to about 8 mm, from about 3 to about 5 mm, or about 4 mm. Housing 201 can have a volume of less than about 1.5 cc, about 1.4 cc, about 1.3 cc, about 1.2 cc, about 1.1 cc, about 1.0 cc, about 0.9 cc, about 0.8 cc, about 0.7 cc, about 0.6 cc, about 0.5 cc, or about 0.4 cc. In some embodiments, housing 201 can have dimensions suitable for implantation through a trocar introducer or any other suitable implantation technique.

As illustrated, electrodes 213 carried by housing 201 are arranged so that all three electrodes 213 do not lie on a common axis. In such a configuration, electrodes 213 can achieve a variety of signal vectors, which may provide one or more improved signals, as compared to electrodes that are all aligned along a single axis. This can be particularly useful in a sensor device 210 configured to be implanted at the neck or head while detecting electrical activity in the brain. In some embodiments, this electrode configuration also provides for improved cardiac ECG sensitivity by integrating 3 potential signal vectors. In some examples, processing circuitry may create virtual signal vectors through a weighted sum or two or more physical signal vectors, such as the physical signal vectors available from electrodes 213 of sensor device 210 or the electrodes of any other sensor device described herein.

In the example shown in FIG. 2B, all electrodes 213 are located on the first major surface 203 and are substantially flat and outwardly facing. However, in other examples one or more electrodes 213 may utilize a three-dimensional configuration (e.g., curved around an edge of the device 210). Similarly, in other examples, such as that illustrated in FIG. 2B, one or more electrodes 213 may be disposed on the second major surface opposite the first. The various electrode configurations allow for configurations in which electrodes 213 are located on both the first major surface and the second major surface. In other configurations, such as that shown in FIG. 2B, electrodes 213 are only disposed on one of the major surfaces of housing 201. Electrodes 213 may be formed of a plurality of different types of biocompatible conductive material (e.g., stainless steel, titanium nitride, platinum iridium, iridium, or alloys thereof), and may utilize one or more coatings such as titanium nitride or fractal titanium nitride. In some embodiments, the material choice for electrodes can also include materials having a high surface area (e.g., to provide better electrode capacitance for better sensitivity) and roughness (e.g., to aid implant stability). Although the example shown in FIGS. 2A and 2B includes four electrodes 213, in some embodiments the sensor device 210 can include 1, 2, 3, 4, 5, 6, or more electrodes carried by housing 201.

FIG. 2C depicts a top view of another example of sensor device 220 in accordance with the present technology. FIG. 2C illustrates sensor device 220 which is substantially similar to sensor device 210, but sensor device 220 includes electrodes 213 which are not exposed along the first major surface 203 of housing 201. Instead, electrodes 213 can be exposed along superior and inferior side surfaces (e.g., facing superiorly and inferiorly when implanted at or on a patient's neck), as shown in FIGS. 2D and 2E. FIG. 2F illustrates sensor device 230 which is substantially similar to sensor devices 210 and 220, but housing 201 is constructed to have a curved configuration, and in which the electrodes can be place along the superior and/or inferior side surfaces of housing 201. In some embodiments, a curved configuration can improve patient comfort and more readily conform to the anatomy of the patient's neck region. In some examples, any of sensor devices 210, 220, or 230 may be flexible in order to conform to the anatomy of the patient at the desired implant or external surface location. Additionally, examples that include electrode extensions, are inherently flexible, allowing conformance to neck and/or cranial anatomy. In some examples, sensor device 220 and/or sensor device 230 may be implanted at a location generally centered with respect to the thorax, the head, e.g., back or temporal regions, neck, or another target region. In some examples, sensor device 220 and/or sensor device 230 may be placed on an external surface of skin of a patient.

In operation, electrodes 213 are used to sense electrical signals (e.g., EEG signals and/or ECG signals) which may be submuscular or subcutaneous. The sensed electrical signals may be stored in a memory of the sensor device 210, and signal data may be transmitted via a communications link to another device (e.g., external device 108 of FIG. 1A). The sensed electrical signals may be time-coded or otherwise correlated with time data, and stored in this form, so that the recency, frequency, time of day, time span, or date(s) of a particular signal data point or data series (or computed measures or statistics based thereon) may be determined and/or reported. In some examples, electrodes 213 may additionally or alternatively be used for sensing any bio-potential signal of interest, such as an electrocardiogram (ECG), intracardiac electrogram (EGM), electromyogram (EMG), or a nerve signal, from any implanted location. These data may be time-coded or time-correlated, and stored in that form, in the manner described above with respect to EEG signal data.

FIG. 2G shows sensor device 270G on the back of a patient's neck. Any of the sensor devices including extensions may be positioned in the manner illustrated by sensor device 270G in FIG. 2G. Additionally, any of the sensor devices may be positioned at other locations described herein, such as temporally as illustrated with respect to FIG. 1B.

Such sensor devices may include one or more extensions extending in a first, inferior direction, toward the neck or shoulders of the patient. Extensions extending in this first direction may position electrodes to facilitate cardiac signal, e.g., ECG, sensing. Such sensor devices may include one or more extensions extending in a second, superior direction, opposite the first direction, toward the upper cranium and scalp of the patient. Extensions extending in this second direction may facilitate brain signal, e.g., EEG, sensing. Each extension may include one or more electrodes to provide one or more sensing vectors of one or more orientations with another electrode on the same extension, a different extension, or a housing of the sensor device.

FIG. 3 depicts an example sensor device 310 in accordance with embodiments of the present technology. In some examples, sensor device 310 can include some or all of the features of IMDs 106 or 400, sensor devices 210, 220, and 230, described herein in accordance with embodiments of the present technology, and can include additional features as described in connection with FIG. 3. In the example shown in FIG. 3, sensor device 310 may be embodied as a monitoring device having housing 314, proximal electrode 313A and distal electrode 313B (individually or collectively “electrode 313” or “electrodes 313”). Housing 314 may further comprise first major surface 318, second major surface 320, proximal end 322, and distal end 324. Housing 314 encloses electronic circuitry located inside sensor device 310 and protects the circuitry contained therein from body fluids. Electrical feedthroughs provide electrical connection of electrodes 313. In an example, sensor device 310 may be embodied as an external monitor, such as patch that may be positioned on an external surface of the patient, or another type of medical device (e.g., instead of as an ICM), such as described further herein.

In the example shown in FIG. 3, sensor device 310 is defined by a length “L,” a width “W,” and thickness or depth “D.” Sensor device 310 may be in the form of an elongated rectangular prism wherein the length L is significantly larger than the width W, which in turn is larger than the depth D. In one example, the geometry of sensor device 310—in particular, a width W being greater than the depth D—is selected to allow sensor device 310 to be inserted under the skin of the patient using a minimally invasive procedure and to remain in the desired orientation during insertion. For example, the device shown in FIG. 3 includes radial asymmetries (notably, the rectangular shape) along the longitudinal axis that maintains the device in the proper orientation following insertion. For example, in one example the spacing between proximal electrode 313a and distal electrode 313B may range from 30 millimeters (mm) to 55 mm, 35 mm to 55 mm, and from 40 mm to 55 mm and may be any range or individual spacing from 25 mm to 60 mm. In-some examples, the length L may be from 30 mm to about 70 mm. In other examples, the length L may range from 40 mm to 60 mm, 45 mm to 60 mm and may be any length or range of lengths between about 30 mm and about 70 mm. In addition, the width W of first major surface 18 may range from 3 mm to 10 mm and may be any single or range of widths between 3 mm and 10 mm. The thickness of depth D of sensor device 310 may range from 2 mm to 9 mm. In other examples, the depth D of sensor device 310 may range from 2 mm to 5 mm and may be any single or range of depths from 2 mm to 9 mm. In addition, sensor device 310 according to an example of the present disclosure is has a geometry and size designed for ease of implant and patient comfort. Examples of sensor device 310 described in this disclosure may have a volume of 3 cc or less, 2 cc or less, 1 cc or less, 0.9 cc or less, 0.8 cc or less, 0.7 cc or less, 0.6 cc or less, 0.5 cc or less, or 0.4 cc or less, any volume between 3 and 0.4 cc, or any volume less than 0.4 cc. In addition, in the example shown in FIG. 3, proximal end 322 and distal end 324 are rounded to reduce discomfort and irritation to surrounding tissue once inserted under the skin of the patient.

In the example shown in FIG. 3, once inserted within the patient, the first major surface 318 faces outward, toward the skin of the patient while the second major surface 320 is located opposite the first major surface 318. Consequently, the first and second major surfaces may face in directions along a sagittal axis of patient, and this orientation may be consistently achieved upon implantation due to the dimensions of sensor device 310. Additionally, an accelerometer, or axis of an accelerometer, may be oriented along the sagittal axis.

Proximal electrode 313A and distal electrode 313B are used to sense electrical signals (e.g., EEG signals, ECG signals, other brain and/or cardiac signals, or impedance) which may be submuscular or subcutaneous. Electrical signals may be stored in a memory of sensor device 310, and signal data may be transmitted via integrated antenna 326 to another medical device, which may be another implantable device or an external device, such as external device 108 (FIG. 1A). In some examples, electrodes 313A and 313B may additionally or alternatively be used for sensing any bio-potential signal of interest, such as an electrocardiogram (ECG), intracardiac electrogram (EGM), electromyogram (EMG), or a nerve signal, from any implanted location.

In the example shown in FIG. 3, proximal electrode 313A is in close proximity to the proximal end 322, and distal electrode 313B is in close proximity to distal end 324. In this example, distal electrode 313B is not limited to a flattened, outward facing surface, but may extend from first major surface 318 around rounded edges 328 or end surface 330 and onto the second major surface 320 so that the electrode 313B has a three-dimensional curved configuration. In the example shown in FIG. 3, proximal electrode 313A is located on first major surface 318 and is substantially flat, outward facing. However, in other examples proximal electrode 313A may utilize the three-dimensional curved configuration of distal electrode 313B, providing a three-dimensional proximal electrode (not shown in this example). Similarly, in other examples distal electrode 313B may utilize a substantially flat, outward facing electrode located on first major surface 318 similar to that shown with respect to proximal electrode 313A. The various electrode configurations allow for configurations in which proximal electrode 313A and distal electrode 313B are located on both first major surface 318 and second major surface 320. In other configurations, such as that shown in FIG. 3, only one of proximal electrode 313A and distal electrode 313B is located on both major surfaces 318 and 320, and in still other configurations both proximal electrode 313A and distal electrode 313B are located on one of the first major surface 318 or the second major surface 320 (e.g., proximal electrode 313A located on first major surface 318 while distal electrode 313B is located on second major surface 320). In another example, sensor device 310 may include electrodes 313 on both first major surface 318 and second major surface 320 at or near the proximal and distal ends of the device, such that a total of four electrodes 313 are included on sensor device 310. Electrodes 313 may be formed of a plurality of different types of biocompatible conductive material (e.g., stainless steel, titanium nitride, platinum, iridium, or alloys thereof), and may utilize one or more coatings such as titanium nitride or fractal titanium nitride. Although the example shown in FIG. 3 includes two electrodes 313, in some embodiments sensor device 310 can include 3, 4, 5, or more electrodes carried by the housing 314.

In the example shown in FIG. 3, proximal end 322 includes a header assembly 332 that includes one or more of proximal electrode 313A, integrated antenna 326, anti-migration projections 334, or suture hole 336. Integrated antenna 326 is located on the same major surface (i.e., first major surface 318) as proximal electrode 313a and is also included as part of header assembly 332. Integrated antenna 326 allows sensor device 310 to transmit or receive data. In other examples, integrated antenna 326 may be formed on the opposite major surface as proximal electrode 313A, or may be incorporated within the housing 314 of sensor device 310. In the example shown in FIG. 3, anti-migration projections 334 are located adjacent to integrated antenna 326 and protrude away from first major surface 318 to prevent longitudinal movement of the device. In the example shown in FIG. 3 anti-migration projections 334 includes a plurality (e.g., six or nine) small bumps or protrusions extending away from first major surface 318. As discussed above, in other examples anti-migration projections 334 may be located on the opposite major surface as proximal electrode 313A or integrated antenna 326. In addition, in the example shown in FIG. 3 header assembly 332 includes suture hole 336, which provides another means of securing sensor device 310 to the patient to prevent movement following insert. In the example shown, suture hole 336 is located adjacent to proximal electrode 313A. In one example, header assembly 332 is a molded header assembly made from a polymeric or plastic material, which may be integrated or separable from the main portion of sensor device 310.

FIG. 4 is a block diagram of an example IMD 400 configured to determine an initial indication of a cranial acute health event and determine whether the initial indication of a cranial acute heath event is a cranial acute health event mimic. IMD 400 may be an example of any of IMD 106 or sensor devices 210, 220, 230, or 310. In the illustrated example, IMD 400 includes electrodes 418A-418C (collectively, “electrodes 418”), antenna 405, processing circuitry 402, sensing circuitry 406, communication circuitry 404, storage device 410, switching circuitry 408, sensors 414 including motion sensor(s) 416, power source 412. In some examples, IMD 400 may further include a clock 419. Although not illustrated in FIG. 4, sensors 414 may include one or more light detectors.

Processing circuitry 402 may include fixed function circuitry and/or programmable processing circuitry. Processing circuitry 402 may include any one or more of a microprocessor, a GPU, a TPU, a controller, a DSP, an ASIC, an FPGA, or equivalent discrete or analog logic circuitry. In some examples, processing circuitry 402 may include multiple components, such as any combination of one or more microprocessors, one or more GPUs, one or more TPUs, one or more controllers, one or more DSPs, one or more ASICs, or one or more FPGAs, as well as other discrete or integrated logic circuitry. The functions attributed to processing circuitry 402 herein may be embodied as software, firmware, hardware or any combination thereof. Processing circuitry 402 may be an example of or component of processing circuitry 110 (FIGS. 1A and 1B), and may be processing circuitry of any of IMD 106 or sensor devices 210, 220, 230, and 310.

Sensing circuitry 406 and communication circuitry 404 may be selectively coupled to electrodes 418A-418C via switching circuitry 408, as controlled by processing circuitry 402. Sensing circuitry 406 may monitor signals from electrodes 418A-418C in order to monitor electrical activity of the brain (e.g., to produce an EEG) and/or heart (e.g., to product an ECG) from which processing circuitry 402 may generate cranial acute health event metrics. Sensing circuitry 406 may also sense physiological characteristics such as subcutaneous tissue impedance, the impedance being indicative of at least some aspects of patient 102's respiratory patterns and the EMG or ECG being indicative of at least some aspects of patient 102's cardiac patterns. Sensing circuitry 406 also may monitor signals from sensors 414, which may include motion sensor(s) 416, and any additional sensors, such as light detectors or optical sensors, pressure sensors, or acoustic sensors, that may be positioned on or in IMD 400.

In some examples, a subcutaneous impedance signal collected by IMD 400 may indicate a respiratory rate and/or a respiratory intensity of patient 102 and an EGM or ECG collected by IMD 400 may indicate a heart rate of patient 102 and an atrial fibrillation (AF) burden of patient 102 or other arrhythmia. In some examples, a respiration component may additionally (using a blended sensor technique) or alternatively be sensed in other signals, such as a motion sensor signal, optical signal, or as a component (e.g., baseline shift) of the cardiac signal sensed via electrodes 418. Sensing circuitry 406 also may monitor signals from sensors 414, which may include motion sensor(s) 416, and any additional sensors, such as light detectors or pressure sensors, that may be positioned on IMD 400. Sensors 414 may also or alternatively detect heart sounds, respiration (e.g., rate or timing), impedance, or blood pressure. Therefore, sensors 414 may also or alternatively include sensors such as one or more microphones, pressure sensors, electrodes, etc. IMD 400 may utilize any of these sensors 414 to determine one or more physiological signals of the patient that may be employed to detect a cranial acute health event. In some examples, sensing circuitry 406 may include one or more filters and amplifiers for filtering and amplifying signals received from one or more of electrodes 418A-418C and/or motion sensor(s) 416. In some examples, sensing circuitry 406 may include separate hardware (e.g., separate circuits) configured to condition and process sensed electrical signals from which cranial acute health event metrics are generated. In this manner each separate circuit may perform one or more filters and amplifiers configured to extract relevant features or signal components from the sensed electrical signals. Moreover, processing circuitry 402 may selectively control each separate circuit depending on whether a cranial acute health event metric should be generated.

Communication circuitry 404 may include any suitable hardware, firmware, software or any combination thereof for communicating with another device, such as external device 108 or another IMD or sensor, such as a pressure sensing device. Under the control of processing circuitry 402, communication circuitry 404 may receive downlink telemetry from, as well as send uplink telemetry to, external device 108 or another device with the aid of an internal or external antenna, e.g., antenna 405. In some examples, communication circuitry 404 may receive downlink telemetry from, as well as send uplink telemetry to, external device 108 or another device via tissue conductance communication (TCC) using two or more of electrodes 418, e.g., as selected by processing circuitry 402 via switching circuitry 408. In addition, processing circuitry 402 may communicate with a networked computing device via an external device (e.g., external device 108) and a computer network, such as the Medtronic CareLink® Network developed by Medtronic, Inc., of Minneapolis, Minnesota. In some examples, communication circuitry 404 may be configured to leverage tissue conductance communication (TCC) for communicating within IMD 400 to between other devices.

A clinician or other user may retrieve data from IMD 400 using external device 108, or by using another local or networked computing device configured to communicate with processing circuitry 402 via communication circuitry 404. The clinician may also program parameters of IMD 400 using external device 108 or another local or networked computing device.

In some examples, storage device 410 may be referred to as a memory and include computer-readable instructions that, when executed by processing circuitry 402, cause IMD 400 and processing circuitry 402 to perform various functions attributed to IMD 400 and processing circuitry 402 herein. Storage device 410 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 410 may also store data generated by sensing circuitry 406, such as physiological information, or data generated by processing circuitry 402, such as cranial acute health event metrics.

Power source 412 is configured to deliver operating power to the components of IMD 400. Power source 412 may include a battery and a power generation circuit to produce the operating power. In some examples, the battery is rechargeable to allow extended operation. In some examples, recharging is accomplished through proximal inductive interaction between an external charger and an inductive charging coil within external device 108. Power source 412 may include any one or more of a plurality of different battery types, such as nickel cadmium batteries and lithium ion batteries. A non-rechargeable battery may be selected to last for several years, while a rechargeable battery may be inductively charged from an external device, e.g., on a daily or weekly basis.

As described herein, IMD 400 may be configured to sense electrical signals and generate cranial acute health event metrics indicative of whether or not patient 102 has experienced a cranial acute health event. In some examples, the processing circuitry 402 is configured to analyze data from one or more electrode combinations using electrodes 418 to extract brain activity data and to discard or reduce any contribution from heart or muscle activity. In some examples, the electrodes 418 are configured to be disposed over the patient's skin. In such embodiments, the electrodes 418 can include protrusions (e.g., microneedles or other suitable structures) configured to at least partially penetrate the patient's skin so as to improve detection of subcutaneous electrical activity.

In some examples, sensing circuitry 406 senses a brain signal via electrodes 418. The brain signal may represent the electrical activity of the brain, and may be an EEG signal. Processing circuitry 402 may determine parameter values from the brain signal, such values determined based on magnitudes of the signal in one or more frequency bands. Sensing circuitry 406 may include filters and other circuitry to isolate the brain signal of interest.

In some examples, sensing circuitry 406 senses a cardiac signal, and processing circuitry 402 may determine parameter values from the cardiac signal. Example parameter values as described herein, such as heart rate or heart rate variability, may be determined based on detection of occurrence of cardiac beats in the cardiac signal. Sensing circuitry 406 may be configured to sense a variety of different signals within which cardiac beats may be identified and values of cardiac parameters may be determined.

For example, sensing circuitry 406 may be configured to sense a cardiac signal representing the electrical activity (e.g., depolarizations and repolarizations) of the heart, such as a subcutaneous ECG signal, via electrodes 418. As another example, sensing circuitry 406 may be configured to sense a cardiac signal representing mechanical activity of the heart via electrodes 418. A component of a signal sensed via electrodes 418, e.g., on or under the scalp of the patient, may vary based on vibration, blood flow, or impedance changes associated with cardiac contractions. Filtering to isolate this component may include 0.5 to 3 Hz bandpass filtering, although other filtering types, ranges, and cutoffs are possible. In some examples, sensing circuitry 406 may be configured to sense a cardiac signal representing mechanical activity of the heart via other sensors 414, such as optical sensors, pressure sensors, or motion sensors 416.

For example, sensing circuitry 406 and/or processing circuitry 402 may detect cardiac pulses via an optical sensor. Processing circuitry 402 may determine heart rate or heart rate variability based on the detection of cardiac pulses via the optical sensor, e.g., in combination with an ECG signal or in the absence of an ECG signal, such as if ECG signal quality is poor. An optical sensor signal may additionally or alternatively be used for other purposes, such as to determine blood oxygenation, local tissue perfusion, or blood pressure, any of which may be useful for detection or prediction of cranial acute health event and/or discrimination of ischemic and hemorrhagic stroke.

One or more electrodes 418 may be positioned, e.g., during implantation of IMD 400, to facilitate sensing of a cardiac signal via the electrodes. In some examples, IMD 400 may include one or more electrode extensions to facilitate positioning of one or more electrodes 418, e.g., via tunneling under the scalp, at desired locations for sensing the brain and/or cardiac signals. Desired locations for sensing brain and cardiac signals using electrodes 418 may be determined prior to implantation of IMD 400 for a particular patient using external sensing equipment, such as standard multi-electrode ECG and EEG equipment, either on the particular patient, or experimentally on a number of subjects. In some examples, the one or more housing-based electrodes 418 of IMD 400 are positioned at a desired location for sensing a brain signal and the one or more extension-based electrodes 418 are positioned at a desired location for sensing a cardiac signal, or vis-a-versa. With reference to FIG. 1D, example locations for positioning an electrode for sensing cardiac signals include P3, PQ3, PQ7, F3, F2, AF3, or C2.

In some examples, processing circuitry 402 may utilize both electrical, e.g., ECG, and pulsatile cardiac signals in an integrated fashion for the detection, prediction, and/or classification of conditions. In some examples, such integration may result in an “enhanced” ECG signal. For example, processing circuitry 402 may identify features within an ECG signal based on the timing of pulses in a pulsatile signal. In some examples, processing circuitry 402 may account for a delay in pulsatile timing relative to the ECG in such integration.

For example, an optical sensor signal (e.g., a photoplethysmographic signal) can be used as a timing base for ensemble averaging or other means to improve the signal-to-noise ratio for a cardiac signal. The optical sensor signal can therefore be considered a surrogate cardiac signal and/or be used to derive an enhanced cardiac signal, which may be particularly useful when the ECG has poor quality. A first or second derivative of an optical sensor signal can be used as a trigger for ensemble averaging, e.g., the ECG signal, by, for example, determining the time associated with a maximum/minimum value of the first or second derivative and/or a zero-crossing of the first or second derivative. Sharp, high-frequency points can be used as trigger points to increase the resolution of the ensemble signal, whereas lower-frequency trigger points may smear or distort the ensemble average. The cardiac waveforms that are aligned with the trigger points can be stored and averaged to generate the ensemble signal.

Processing circuitry 402 may be configured to calculate physiological characteristics relating to one or more electrical signals received from the electrodes 418, such as cranial acute health event metrics. For example, processing circuitry 402 may be configured to algorithmically determine the presence or absence of a cranial acute health event (via generation of a cranial acute health event metric) or other neurological condition from the electrical signal. In certain examples, processing circuitry 402 may make a cranial acute health event determination for each electrode 418 (e.g., channel) or may make a cranial acute health event determination using electrical signals acquired from two or more selected electrodes 418.

In some examples, processing circuitry 402 may employ patient movement information as a part of cranial acute health event detection. For example, motion sensor 416 may include one or more accelerometers discussed above. In some examples, motion sensors 416 may be configured to detect patient posture, patient activity, and/or patient movement, which includes detection of patient falling.

In some examples, sensing circuitry 406 senses electrical signals from the patient. Sensing circuitry 406 may sense these electrical signals from a sensing vector determined by the electrodes 418 selected for sensing. In this manner, sensing circuitry 406 may use different vectors (e.g., different electrode combinations) in order to obtain different electrical information from the patient. Sensing circuitry 406 may generate one or more EEG signals based on the sensed electrical signals. Generating one or more EEG signals may include various filtering, amplification, transforms, digitization, or any other conditioning and processing that generates an EEG signal can be analyzed by processing circuitry 402.

In some examples, processing circuitry 402 may receive the one or more EEG signals from sensing circuitry 406 during a first period of time and determine an initial indication of a cranial acute health event during the first period of time based on at least one particular feature of the one or more EEG signals generated during the first period of time. In some examples, processing circuitry 402 may determine the at least one particular feature of the one or more EEG signals generated during the first period of time based on the one or more EEG signals generated during the first period of time. In some examples, the at least one particular feature comprises a bandwidth characteristic of the one or more EEG signals. In some examples, the bandwidth characteristic includes a bandwidth ratio. In some examples, the bandwidth ratio includes a DAR. In some examples, the cranial acute health event includes a stroke.

In some examples, processing circuitry 402 may receive the one or more EEG signals from sensing circuitry 406 during a second period of time. In some examples, processing circuitry 402 may determine whether difference(s) between the at least one particular feature of the one or more EEG signals generated during the first period of time and at least one corresponding particular feature of the one or more EEG signals generated during the second period of time satisfy a cranial acute health event mimic threshold. In some examples, processing circuitry 402 may determine the at least one corresponding particular feature of the one or more EEG signals generated during the second period of time based on the one or more EEG signals generated during the second period of time. In some examples, the second period of time is after the first period of time. In some examples, the second period of time does not overlap with the first period of time. In some examples, processing circuitry 402 may determine the at least one corresponding particular feature of the one or more EEG signals generated during the second period at an end of the second period of time.

In some examples, in response to the difference(s) satisfying the cranial acute health event mimic threshold, processing circuitry 402 may determine the initial indication of cranial acute health event during the first period of time is a cranial acute health event mimic. In some examples, in response to the difference(s) not satisfying the cranial acute health event mimic threshold processing circuitry 402 may verify the initial indication of cranial acute health event during the first period of time is a cranial acute health event. In some examples, processing circuitry 402 may determine the difference(s) satisfy the cranial acute health event mimic threshold based on a value of the difference(s) being greater than the cranial acute health event mimic threshold. In some examples, a cranial acute health event is a stroke.

In some examples, processing circuitry 402 may receive one or more EEG signals of a first brain hemisphere from sensing circuitry 406 and determine an initial indication of a cranial acute health event based on at least one particular feature of the one or more EEG signals of the first brain hemisphere. In some examples, processing circuitry 402 may determine the at least one particular feature of the one or more EEG signals of the first brain hemisphere. In some examples, processing circuitry 402 may determine the at least one particular feature of the one or more EEG signals of the first brain hemisphere during a first period of time. In some examples, the at least one particular feature comprises a bandwidth characteristic of the one or more EEG signals. In some examples, the bandwidth characteristic includes a bandwidth ratio. In some examples, the bandwidth ratio includes a DAR. In some examples, the cranial acute health event includes a stroke.

In some examples, processing circuitry 402 may receive one or more EEG signals of a second brain hemisphere from sensing circuitry 406. In some examples, processing circuitry 402 may determine whether difference(s) between the at least one particular feature of the one or more EEG signals of the first brain hemisphere and at least one corresponding particular feature of the one or more EEG signals of the second brain hemisphere satisfy a cranial acute health event brain hemisphere mimic threshold. In some examples, processing circuitry 402 may determine the at least one particular feature of the one or more EEG signals of the second brain hemisphere. In some examples, processing circuitry 402 may determine the at least one particular feature of the one or more EEG signals of the second brain hemisphere during a second period of time. In some examples, the second period of time is after the first period of time. In some examples, the first brain hemisphere is a first side brain hemisphere (e.g., a left side brain hemisphere or a right side brain hemisphere) and the second brain hemisphere is an opposite side brain hemisphere (e.g., if the first brain hemisphere is a left side brain hemisphere, the second brain hemisphere is a right side brain hemisphere).

In some examples, processing circuitry 402 may determine the difference(s) satisfy the cranial acute health event brain hemisphere mimic threshold based on a value of the difference(s) being less than the cranial acute health event brain hemisphere mimic threshold. In some examples, processing circuitry 402 may determine the difference(s) do not satisfy the cranial acute health event brain hemisphere mimic threshold based on a value of the difference(s) being greater than the cranial acute health event brain hemisphere mimic threshold.

In some examples, in response to the difference(s) satisfying the cranial acute health event brain hemisphere mimic threshold, processing circuitry 402 may determine the initial indication of cranial acute health event is a cranial acute health event mimic. In some examples, in response to the difference(s) not satisfying the cranial acute health event brain hemisphere mimic threshold, processing circuitry 402 may verify the initial indication of cranial acute health event is a cranial acute health event.

In some examples, if processing circuitry 402 does not have instructions to transmit the metric information to an external device (such as external device 108) because processing circuitry 402 determines the initial indication of cranial acute health event is cranial acute health event mimic, processing circuitry 402 may continue to sense electrical signals from the patient.

In some examples, if processing circuitry 402 has instructions to transmit the metric information to an external device (such as external device 108) because processing circuitry 402 verifies the initial indication of cranial acute health event is a cranial acute health event, such as a stroke or a particular type of stroke, processing circuitry 402 may transmit an indication of a cranial acute health event and/or the at least one particular features of the one or more EEG signals to an external device (such as external device 108). In some examples, processing circuitry 402 may also continue to sense electrical signals from the patient.

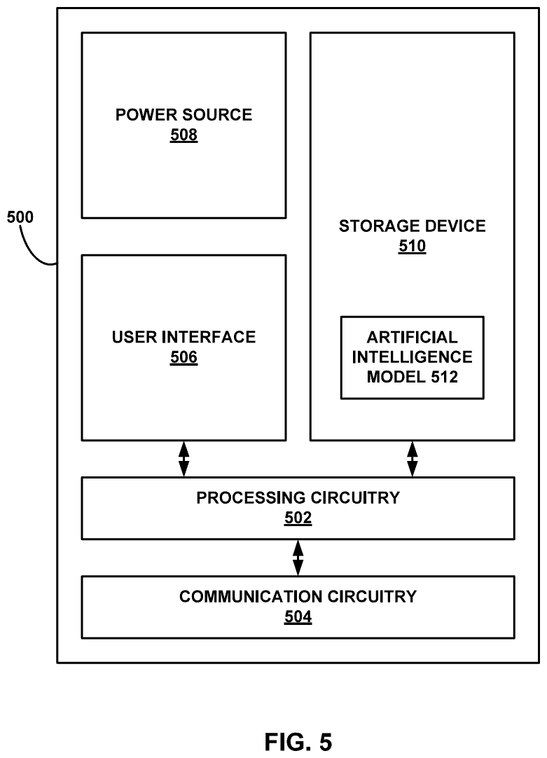

FIG. 5 is a block diagram of an example external device 500 configured to communicate with any IMD (e.g., IMD 106 or IMD 400) or sensor device described herein. External device 500 is an example of external device 108 of FIG. 1A. In the example of FIG. 5, external device 500 includes processing circuitry 502, communication circuitry 504, storage device 510, user interface 506, and power source 508.

Processing circuitry 502, in one example, may include one or more processors that are configured to implement functionality and/or process instructions for execution within external device 500. For example, processing circuitry 502 may be capable of processing instructions stored in storage device 510. Processing circuitry 502 may include, for example, microprocessors, GPUs, TPUs, DSPs, ASICs, FPGAs, or equivalent discrete or integrated logic circuitry, or a combination of any of the foregoing devices or circuitry. Accordingly, processing circuitry 502 may include any suitable structure, whether in hardware, software, firmware, or any combination thereof, to perform the functions ascribed herein to processing circuitry 502.

Communication circuitry 504 may include any suitable hardware, firmware, software or any combination thereof for communicating with another device, such as IMD 400. Under the control of processing circuitry 502, communication circuitry 504 may receive downlink telemetry from, as well as send uplink telemetry to, IMD 400, or another device. In other examples, communication circuitry 504 may also employ TCC for communicating with other devices.

Storage device 510 may be configured to store information within external device 500 during operation. Storage device 510 may include a computer-readable storage medium or computer-readable storage device. In some examples, storage device 510 includes one or more of a short-term memory or a long-term memory. Storage device 510 may include, for example, RAM, dynamic random access memories (DRAM), static random access memories (SRAM), magnetic discs, optical discs, flash memories, or forms of electrically programmable memories (EPROM) or EEPROM. In some examples, storage device 510 is used to store data indicative of instructions for execution by processing circuitry 502. Storage device 510 may be used by software or applications running on external device 500 to temporarily store information during program execution. In some examples, storage device 510 may include an artificial intelligence model 512 (e.g., machine learning, neural networks, etc.) stored on the storage device 510.

Data exchanged between external device 500 and IMD 400 may include operational parameters. External device 500 may transmit data including computer readable instructions which, when implemented by IMD 400, may control IMD 400 to change one or more operational parameters and/or export collected data. For example, processing circuitry 502 may transmit an instruction to IMD 400 which requests IMD 400 to export collected data (e.g., data corresponding to one or more of the EEG signals, one or more particular features of an EEG signal, and/or an indication of cranial acute health event) to external device 500. In turn, external device 500 may receive the collected data from IMD 400 and store the collected data in storage device 510. Additionally, or alternatively, processing circuitry 502 may export instructions to IMD 400 requesting IMD 400 to update electrode combinations for sensing.

A user, such as a clinician or patient 102, may interact with external device 500 through user interface 506. User interface 506 includes a display (not shown), such as an LCD or LED display or other type of screen, with which processing circuitry 502 may present information related to IMD 400 (e.g., determination an initial indication of cranial acute health event is a cranial acute health event mimic or verification of a cranial acute health event). In addition, user interface 506 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 502 of external device 500 and provide input. In other examples, user interface 506 also includes audio circuitry for providing audible notifications, instructions or other sounds to patient 102, receiving voice commands from patient 102, or both. Storage device 510 may include instructions for operating user interface 506 and for managing power source 508.

Power source 508 is configured to deliver operating power to the components of external device 500. Power source 508 may include a battery and a power generation circuit to produce the operating power. In some examples, the battery is rechargeable to allow extended operation. Recharging may be accomplished by electrically coupling power source 508 to a cradle or plug that is connected to an alternating current (AC) outlet. In addition, recharging may be accomplished through proximal inductive interaction between an external charger and an inductive charging coil within external device 500. In other examples, traditional batteries (e.g., nickel cadmium or lithium ion batteries) may be used. In addition, external device 500 may be directly coupled to an alternating current outlet to operate.

In some examples, external device 500 may provide an alert to the patient or another entity (e.g., a call center) based on a cranial acute health event indication/verification provided by IMD 400. In some examples, user interface 506 may provide an interface for presenting an alert of the detection, prediction, or classification of the condition, e.g., stroke, and for a user, e.g., the patient, a caregiver, or a clinician, to provide input overriding the detection, prediction, or classification. In this manner, systems as described herein may avoid unnecessary emergency activity resulting from a false detection by the system such as avoiding unnecessary emergency activity resulting from reporting a cranial acute health event mimic as a cranial acute health event.

In some examples, external device 500 may receive patient data, such as one or more of the EEG signals, such as from IMD 400, to further adjudicate whether difference(s) between particular features of EEG signal(s) satisfy a cranial acute health event mimic threshold in IMD 400. For example, external device 500 may confirm or deny whether an initial indication of a cranial acute health event detected by IMD 400 is a true cranial acute health event. In some examples, external device 500 may further confirm or deny what type of cranial acute health event was detected by IMD 400. In some examples, external device may apply an artificial intelligence model 512 (e.g., machine learning, neural networks, etc.) stored in storage device 510 to the received patient data to confirm or deny whether an initial indication of a cranial acute health event detected by IMD 400 is a true cranial acute health event and/or confirm or deny what type of cranial acute health event was detected by IMD 400.

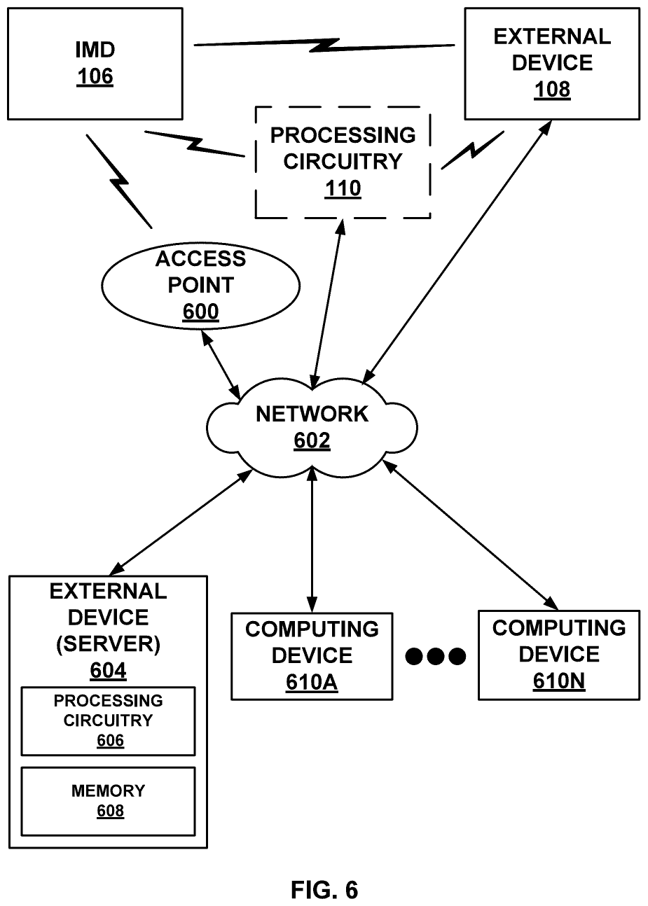

FIG. 6 is a block diagram illustrating an example system that includes an access point 600, a network 602, external computing devices, such as a server 604, and one or more other computing devices 610A-610N, which may be coupled to IMD 106, external device 108, and processing circuitry 110 via network 602, in accordance with one or more techniques described herein. In this example, IMD 106 may use communication circuitry to communicate with external device 108 via a first wireless connection, and to communicate with an access point 600 via a second wireless connection. In the example of FIG. 6, access point 600, external device 108, server 604, and computing devices 610A-610N are interconnected and may communicate with each other through network 602.

Access point 600 may include a device that connects to network 602 via any of a variety of wired or wireless network connections, such as telephone dial-up, digital subscriber line (DSL), or cable modem connections. In other examples, access point 600 may be coupled to network 602 through different forms of connections, including wired or wireless connections. In some examples, access point 600 may be a user device, such as a tablet or smartphone, that may be co-located with the patient. As discussed above, IMD 106 may be configured to transmit data, such as any one or combination of an EEG signal, an accelerometer signal, and a tissue impedance signal to external device 108. In addition, access point 600 may interrogate IMD 106, such as periodically or in response to a command from the patient or network 602, in order to retrieve parameter values determined by processing circuitry of IMD 106, or other operational or patient data from IMD 106. Access point 600 may then communicate the retrieved data to server 604 via network 602.

In some cases, server 604 may be configured to provide a secure storage site for data that has been collected from IMD 106, and/or external device 108. In some cases, server 604 may assemble data in web pages or other documents for viewing by trained professionals, such as clinicians, via computing devices 610A-610N. One or more aspects of the illustrated system of FIG. 6 may be implemented with general network technology and functionality, which may be similar to that provided by the Medtronic CareLink® Network developed by Medtronic plc, of Dublin, Ireland.

Server 604 may include processing circuitry 606. Processing circuitry 606 may include fixed function circuitry and/or programmable processing circuitry. Processing circuitry 606 may include any one or more of a microprocessor, a GPU, a TPU, a controller, a DSP, an ASIC, an FPGA, or equivalent discrete or analog logic circuitry. In some examples, processing circuitry 606 may include multiple components, such as any combination of one or more microprocessors, one or more controllers, one or more DSPs, one or more ASICs, or one or more FPGAs, as well as other discrete or integrated logic circuitry. The functions attributed to processing circuitry 606 herein may be embodied as software, firmware, hardware or any combination thereof. In some examples, processing circuitry 606 may perform one or more techniques described herein based on an EEG signal, EGM signal, impedance signal, an accelerometer signal, or other sensor signals received from IMD 106, or parameter values determined based on such signals by IMD 106 and received from IMD 106, as examples. For example, processing circuitry 606 may perform one or more of the techniques described herein to determine an initial indication of a cranial acute health event and determine/verify whether the initial indication of a cranial acute health event is a cranial acute health event mimic or is a cranial acute health event.

Server 604 may include memory 608. Memory 608 includes computer-readable instructions that, when executed by processing circuitry 606, cause IMD 106 and processing circuitry 606 to perform various functions attributed to IMD 106 and processing circuitry 606 herein. Memory 608 may include any volatile, non-volatile, magnetic, optical, or electrical media, such as RAM, ROM, NVRAM, EEPROM, flash memory, or any other digital media.

In some examples, one or more of computing devices 610A-610N (e.g., device 610A) may be a tablet or other smart device located with a clinician, by which the clinician may program, receive alerts from, and/or interrogate IMD 106. For example, the clinician may access data corresponding to any one or combination of sensed physiological signals, EEG signal(s), particular features of EEG signals, indication of a cranial acute health event, determination that an indication of a cranial acute health event is a cranial acute health event mimic, verification that an indication of a cranial acute health event is a cranial acute health event, and other types of signals collected by IMD 106, or parameter values determined by IMD 106 based on such signals, through device 610A, such as when patient 102 is in between clinician visits, to check on a status of a medical condition, such as sensed EEG signals, particular features of EEG signals, an indication of a cranial acute health event, verification that an indication of a cranial acute health event is a cranial acute health event, and/or determination that an indication of a cranial acute health event is a cranial acute health event mimic. In some examples, the clinician may enter instructions for a medical intervention for patient 102 into an app in device 610A, such as based on a status of a patient condition determined by IMD 106, external device 108, processing circuitry 110, or any combination thereof, or based on other patient data known to the clinician. Device 610A then may transmit the instructions for medical intervention to another of computing devices 610A-610N (e.g., device 610B) located with patient 102 or a caregiver of patient 102. For example, such instructions for medical intervention may include an instruction to change a drug dosage, timing, or selection, to schedule a visit with the clinician, or to seek medical attention. In further examples, device 610B may generate an alert to patient 102 based on a status of a medical condition of patient 102 determined by IMD 106, which may enable patient 102 proactively to seek medical attention prior to receiving instructions for a medical intervention. In this manner, patient 102 may be empowered to take action, as needed, to address his or her medical status, which may help improve clinical outcomes for patient 102.

In further examples, device 610B may be configured to transmit alert messages to computing devices 610C associated with one or more care providers via network 602. Care providers may include emergency medical systems (EMS) and hospitals, and may include particular departments within a hospital, such as an emergency department, catheterization lab, or a stroke response department. In further examples, device 610B may provide patient-specific care recommendations (e.g., potential drug therapy for prevention or therapy of cranial acute health event). The ability of the system to detect the cranial acute health event with adequate sensitivity and specificity may, for example, guide EMS care giver to what they can expect when they arrive on the scene and how best to treat the presenting or soon to present cranial acute health event.

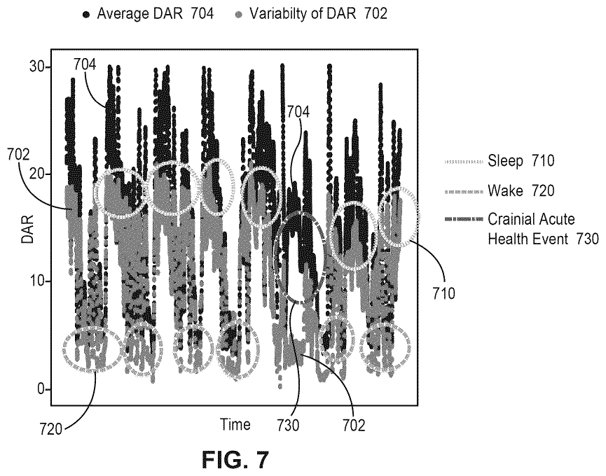

FIG. 7 is an example graph of differences between particular features of EEG signal(s) that satisfy a cranial acute health mimic threshold or a cranial acute health event brain hemisphere mimic threshold. Processing circuitry 402 of IMD 400 will be described as performing the techniques of example FIG. 7, but other components, devices, and systems (e.g., IMD 106 or sensor devices 210, 220, or 230) may perform similar functionality in other examples.

FIG. 7 shows a graph of average DAR 704 of EEG signal(s) over a period of time and variability of DAR 704, such as standard deviation, of EEG signal(s) over a period of time. As shown in FIG. 7, low values of average DAR 704 and variability of DAR 702 occur while a person is awake 720, and an increased average DAR 704 and increased variability of DAR 702, such as due to sleep cycles, may occur while a person is sleeping 710. In some examples, processing circuitry 402 may determine particular features of EEG signal(s) satisfy a cranial acute health mimic threshold or a cranial acute health event brain hemisphere mimic threshold when both the average DAR 704 satisfies an average DAR threshold, and the variability of DAR 702 satisfies a DAR variability threshold. For example, in response to the difference satisfying the cranial acute health event mimic threshold, processing circuitry 402 may determine an initial indication of cranial acute health event is a cranial acute health event mimic (e.g., patient is sleeping).

In some examples, processing circuitry 402 may determine particular features of EEG signal(s) do not satisfy a cranial acute health mimic threshold or a cranial acute health event brain hemisphere mimic threshold when the average DAR 704 satisfies an average DAR threshold, and the variability of DAR 702 does not satisfy a DAR variability threshold. For example, when the average DAR 704 is high (e.g., satisfies an average DAR threshold), as shown in 730, and the variability of DAR 702 is low (e.g., does not satisfy a DAR variability threshold), as shown in 730, processing circuitry 402 is configured to, based on the average DAR 704 and the DAR variability 702 of the EEG signal(s), verify an initial indication of cranial acute health event, such as stroke, is a cranial acute health event, such as a stroke.

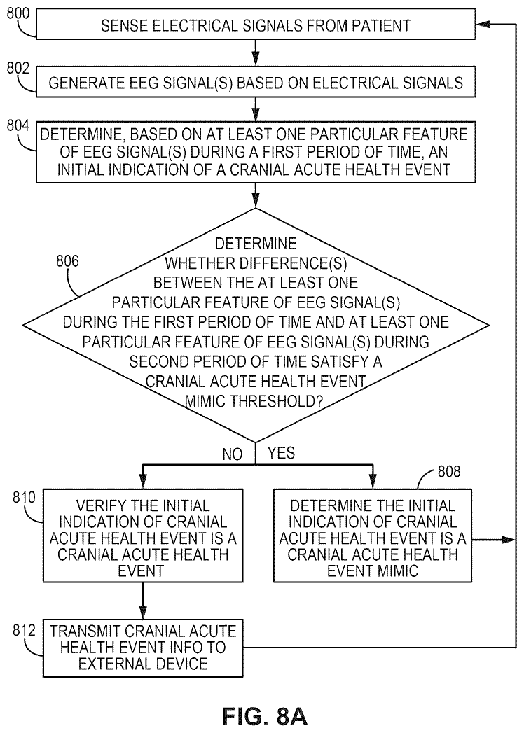

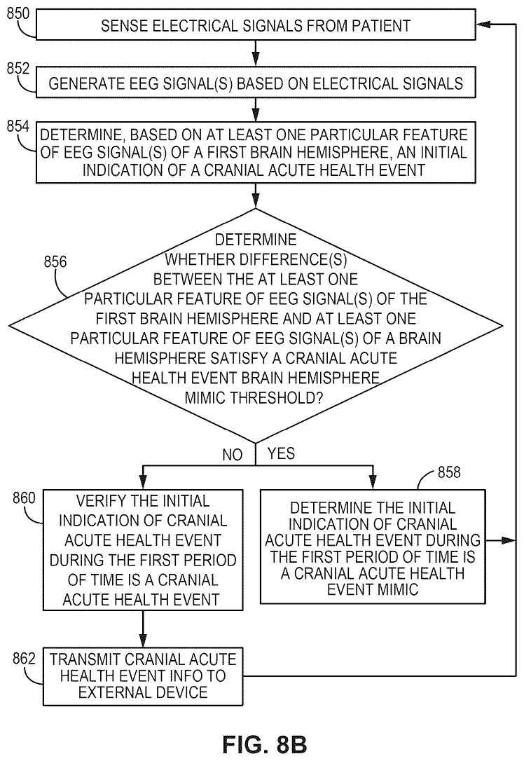

FIG. 8A is a flow diagram of an example technique for determining whether a cranial acute health event, such as a stroke, occurred in the patient. Sensing circuitry 406 and processing circuitry 402 of IMD 400 will be described as performing the example techniques of FIG. 8A, but other components, devices, and systems (e.g., IMD 106 or sensor devices 210, 220, or 230) may perform similar functionality in other examples. As shown in the example of FIG. 8A, sensing circuitry 406 senses electrical signals from the patient (800). Sensing circuitry 406 may sense these electrical signals from a sensing vector determined by the electrodes 418 selected for sensing. In this manner, sensing circuitry 406 may use different vectors (e.g., different electrode combinations) in order to obtain different electrical information from the patient. Sensing circuitry 406 may then generate one or more EEG signals based on the sensed electrical signals (802). Generating one or more EEG signals may include various filtering, amplification, transforms, digitization, or any other conditioning and processing that generates an EEG signal can be analyzed by processing circuitry 402.

Processing circuitry 402 is configured to receive one or more EEG signals from sensing circuitry 406, such as one or more EEG signals during a first period of time and/or one or more EEG signals during a second period of time. Processing circuitry may determine, based on at least one particular feature of the one or more EEG signals generated during the first period of time, an initial indication of a cranial acute health event during the first period of time (804). In some examples, processing circuitry 402 may determine the at least one particular feature of the one or more EEG signals generated during the first period of time based on the one or more EEG signals generated during the first period of time. In some examples, the at least one particular feature comprises a bandwidth characteristic of the one or more EEG signals. In some examples, the bandwidth characteristic includes a bandwidth ratio. In some examples, the bandwidth ratio includes a DAR. In some examples, the cranial acute health event includes a stroke.

Processing circuitry 402 may determine whether difference(s) between at least one particular feature of the one or more EEG signals generated during the first period of time and at least one corresponding particular feature of the one or more EEG signals generated during the second period of time satisfy a cranial acute health event mimic threshold (806). In some examples, processing circuitry 402 may determine the at least one corresponding particular feature of the one or more EEG signals generated during the second period of time based on the one or more EEG signals generated during the second period of time. In some examples, the second period of time is after the first period of time. In some examples, the second period of time does not overlap with the first period of time. In some examples, processing circuitry 402 may determine the at least one corresponding particular feature of the one or more EEG signals generated during the second period at an end of the second period of time. In some examples, the at least one corresponding particular feature comprises a bandwidth characteristic of the one or more EEG signals. In some examples, the bandwidth characteristic includes a bandwidth ratio. In some examples, the bandwidth ratio includes a DAR. In some examples, the cranial acute health event includes a stroke.

If processing circuitry 402 determines the difference(s) satisfy the cranial acute health event mimic threshold (“YES” branch of block 806), processing circuitry 402 may determine the initial indication of cranial acute health event is a cranial acute health event mimic (808). In some examples, processing circuitry 402 may determine the difference(s) satisfy the cranial acute health event mimic threshold based on value(s) of the difference(s) being greater than the cranial acute health event mimic threshold. In some examples, a cranial acute health event is a stroke.

If processing circuitry 402 determines the difference(s) do not satisfy the cranial acute health event mimic threshold (“NO” branch of block 806), processing circuitry 402 may verify the initial indication of cranial acute health event during the first period of time is a cranial acute health event (810). In some examples, processing circuitry 402 may determine the difference(s) do not satisfy the cranial acute health event mimic threshold based on value(s) of the difference(s) being less than the cranial acute health event mimic threshold.

In some examples, processing circuitry 402 may transmit an indication of the verified cranial acute health event and/or information related to the verified cranial acute health event (812), such as at least one particular features of the one or more EEG signals, to an external device (such as external device 108) because processing circuitry 402 verifies the initial indication of cranial acute health event is a cranial acute health event, such as a stroke or a particular type of stroke. In some examples, processing circuitry 402 may also continue to sense electrical signals from the patient (800), such as after transmitting an indication of the verified cranial acute health event and/or information related to the verified cranial acute health event to an external device.

In some examples, after processing circuitry 402 determines the initial indication of cranial acute health event is a cranial acute health event mimic, processing circuitry 402 may continue to sense electrical signals from the patient (800).

FIG. 8B is a flow diagram of an example technique for determining whether a cranial acute health event, such as a stroke, occurred in the patient. Sensing circuitry 406 and processing circuitry 402 of IMD 400 will be described as performing the example techniques of FIG. 8B, but other components, devices, and systems (e.g., IMD 106 or sensor devices 210, 220, or 230) may perform similar functionality in other examples. As shown in the example of FIG. 8B, sensing circuitry 406 senses electrical signals from the patient (850). Sensing circuitry 406 may sense these electrical signals from a sensing vector determined by the electrodes 418 selected for sensing. In this manner, sensing circuitry 406 may use different vectors (e.g., different electrode combinations) in order to obtain different electrical information from the patient. Sensing circuitry 406 may then generate one or more EEG signals based on the sensed electrical signals (852). Generating one or more EEG signals may include various filtering, amplification, transforms, digitization, or any other conditioning and processing that generates an EEG signal can be analyzed by processing circuitry 402.

Processing circuitry 402 is configured to receive one or more EEG signals from sensing circuitry 406, such as one or more EEG signals of a first brain hemisphere and/or one or more EEG signals of a second brain hemisphere. Processing circuitry may determine, based on at least one particular feature of the one or more EEG signals of the first brain hemisphere, an initial indication of a cranial acute health event (854). In some examples, processing circuitry 402 may determine the at least one particular feature of the one or more EEG signals of the first brain hemisphere. In some examples, processing circuitry 402 may determine the at least one particular feature of the one or more EEG signals of the first brain hemisphere during a first period of time. In some examples, the at least one particular feature comprises a bandwidth characteristic of the one or more EEG signals. In some examples, the bandwidth characteristic includes a bandwidth ratio. In some examples, the bandwidth ratio includes a DAR. In some examples, the cranial acute health event includes a stroke.

Processing circuitry 402 may determine whether difference(s) between at least one particular feature of the one or more EEG signals of the first brain hemisphere and at least one corresponding particular feature of the one or more EEG signals of a second brain hemisphere satisfy a cranial acute health event brain hemisphere mimic threshold (856). In some examples, processing circuitry 402 may determine the at least one particular feature of the one or more EEG signals of the second brain hemisphere. In some examples, processing circuitry 402 may determine the at least one particular feature of the one or more EEG signals of the second brain hemisphere during a second period of time. In some examples, the second period of time is after the first period of time. In some examples, the second period of time does not overlap with the first period of time. In some examples, the first brain hemisphere is a first side brain hemisphere (e.g., a left side brain hemisphere or a right side brain hemisphere) and the second brain hemisphere is an opposite side brain hemisphere (e.g., if the first brain hemisphere is a left side brain hemisphere, the second brain hemisphere is a right side brain hemisphere).

If processing circuitry 402 determines the difference(s) satisfy the cranial acute health event brain hemisphere mimic threshold (“YES” branch of block 856), processing circuitry 402 may determine the initial indication of cranial acute health event is a cranial acute health event mimic (858). In some examples, processing circuitry 402 may determine the differences satisfy the cranial acute health event brain hemisphere mimic threshold based on a value of the differences being less than the cranial acute health event brain hemisphere mimic threshold. In some examples, a cranial acute health event is a stroke.

If processing circuitry 402 determines the difference(s) do not satisfy the cranial acute health event brain hemisphere mimic threshold (“NO” branch of block 856), processing circuitry 402 may verify the initial indication of cranial acute health event is a cranial acute health event (860). In some examples, processing circuitry 402 may determine the differences do not satisfy the cranial acute health event brain hemisphere mimic threshold based on a value of the differences being greater than the cranial acute health event brain hemisphere mimic threshold.

In some examples, processing circuitry 402 may transmit an indication of the verified cranial acute health event and/or information related to the verified cranial acute health event, such as at least one particular features of the one or more EEG signals (862), to an external device (such as external device 108) because processing circuitry 402 verifies the initial indication of cranial acute health event is a cranial acute health event, such as a stroke or a particular type of stroke. In some examples, processing circuitry 402 may also continue to sense electrical signals from the patient (850), such as after transmitting an indication of the verified cranial acute health event and/or information related to the verified cranial acute health event to an external device.

In some examples, after processing circuitry 402 determines the initial indication of cranial acute health event is a cranial acute health event mimic, processing circuitry 402 may continue to sense electrical signals from the patient (850).

As described herein the cranial acute health event indication, such as a stroke indication, can be, for example, a binary output of cranial acute health event condition/non-cranial acute health event condition, a probabilistic indication of cranial acute health event likelihood, or other output relating to the patient's condition and likelihood of having suffered a cranial acute health event. In some examples, the cranial acute health event indication may be a metric that can be calculated using a classifier model as described elsewhere herein.

When processing circuitry 402 transmits the stroke metric to an external device, the external device may be associated with emergency services in some examples. In some examples, the external device may include global position system (GPS) capability or other location detection technology (e.g., WiFi triangulation) such that the external device can identify, store, and/or communicate the geographic location at which the stroke metric occurred. The external device may then transmit the location information and/or stroke metric to another device or system via cell phone tower, satellite, or other technology. The other system may be an emergency service such as 911 or other medical service. If one or more of the techniques of FIGS. 8A-8B are performed in an ambulance, for example, a device carried by the ambulance or technician may receive the indication and output information or instructions to an emergency medical technician (EMT) or other personnel in the rear of the ambulance and/or to the ambulance driver. In some embodiments, the display to the ambulance driver can include navigational information such as a map and instructions to take the patient to a particular hospital or facility with a stroke center.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques described herein.

Example 1: A system includes a plurality of electrodes; sensing circuitry configured to: sense, via at least two electrodes of the plurality of electrodes, electrical signals from a patient; and generate, based on the electrical signals, one or more electroencephalography (EEG) signals; and processing circuitry configured to: receive, from the sensing circuitry, one or more EEG signals generated during a first period of time; determine, based on at least one particular feature of the one or more EEG signals generated during the first period of time, an initial indication of a cranial acute health event during the first period of time; receive, from the sensing circuitry, one or more EEG signals generated during a second period of time; determine whether a difference between the at least one particular feature of the one or more EEG signals generated during the first period of time and at least one corresponding particular feature of the one or more EEG signals generated during the second period of time satisfies a cranial acute health event mimic threshold; in response to the difference satisfying the cranial acute health event mimic threshold, determine the initial indication of cranial acute health event during the first period of time is a cranial acute health event mimic; and in response to the difference not satisfying the cranial acute health event mimic threshold, verify the initial indication of cranial acute health event during the first period of time is a cranial acute health event.

Example 2: The system of example 1, wherein the cranial acute health event is a stroke.

Example 3: The system of any of examples 1-2, wherein the at least one particular feature comprises a bandwidth characteristic of the one or more EEG signals.

Example 4: The system of example 3, wherein the bandwidth characteristic includes a bandwidth ratio.

Example 5: The system of example 4, wherein the bandwidth ratio includes a delta-alpha ratio.

Example 6: The system of any of examples 1-5, wherein the second period of time does not overlap with the first period of time.

Example 7: The system of any of examples 1-5, wherein the second period of time is after the first period of time.

Example 8: The system of any of examples 1-7, wherein the processing circuitry is further configured to: determine, based on the one or more EEG signals generated during the first period of time, the at least one particular feature of the one or more EEG signals generated during the first period of time; and determine, based on the one or more EEG signals generated during the second period of time, the at least one corresponding particular feature of the one or more EEG signals generated during the second period of time.

Example 9: The system of example 8, wherein the processing circuitry is further configured to determine the at least one corresponding particular feature of the one or more EEG signals generated during the second period at an end of the second period of time.

Example 10: The system of any of examples 1-9, wherein the processing circuitry is further configured to determine the difference satisfies the cranial acute health event mimic threshold based on a value of the difference being greater than the cranial acute health event mimic threshold.

Example 11: A system includes a plurality of electrodes; sensing circuitry configured to: sense, via at least two electrodes of the plurality of electrodes, electrical signals from a patient; and generate, based on the electrical signals, one or more electroencephalography (EEG) signals; and processing circuitry configured to: receive, from the sensing circuitry, one or more EEG signals of a first brain hemisphere; determine, based on at least one particular feature of the one or more EEG signals of the first brain hemisphere, an initial indication of a cranial acute health event; receive, from the sensing circuitry, one or more EEG signals of a second brain hemisphere; determine whether a difference between the at least one particular feature of the one or more EEG signals of the first brain hemisphere and at least one corresponding particular feature of the one or more EEG signals of the second brain hemisphere satisfies a cranial acute health event brain hemisphere mimic threshold; in response to the difference satisfying the cranial acute health event brain hemisphere mimic threshold, determine the initial indication of cranial acute health event is a cranial acute health event mimic; and in response to the difference not satisfying the cranial acute health event brain hemisphere mimic threshold, verify the initial indication of cranial acute health event is a cranial acute health event.

Example 12: The system of example 11, wherein the cranial acute health event is a stroke.

Example 13: The system of any of examples 11-12, wherein the at least one particular feature comprises a bandwidth characteristic of the one or more EEG signals.

Example 14: The system of example 13, wherein the bandwidth characteristic includes a bandwidth ratio.

Example 15: The system of example 14, wherein the bandwidth ratio includes a delta-alpha ratio.

Example 16: The system of any of examples 11-15, wherein the processing circuitry is further configured to: determine the at least one particular feature of the one or more EEG signals of the first brain hemisphere; and determine the at least one particular feature of the one or more EEG signals of the second brain hemisphere.

Example 17: The system of example 16, wherein the processing circuitry is further configured to: determine the at least one particular feature of the one or more EEG signals of the first brain hemisphere during a first period of time; and determine the at least one particular feature of the one or more EEG signals of the second brain hemisphere during a second period of time, wherein the second period of time is after the first period of time.

Example 18: The system of any of examples 11-17, wherein the processing circuitry is further configured to determine the difference satisfies the cranial acute health event brain hemisphere mimic threshold based on a value of the difference being less than the cranial acute health event brain hemisphere mimic threshold.

Example 19: The system of any of examples 11-17, wherein the processing circuitry is further configured to determine the difference do not satisfy the cranial acute health event brain hemisphere mimic threshold based on a value of the difference being greater than the cranial acute health event brain hemisphere mimic threshold.

Example 20: The system of any of examples 11-19, wherein the first brain hemisphere is a first side brain hemisphere and the second brain hemisphere is an opposite side brain hemisphere.

Example 21: A method includes sensing, by sensing circuitry and via at least two electrodes of a plurality of electrodes, electrical signals from a patient; generating, by the sensing circuitry and based on the electrical signals, one or more electroencephalography (EEG) signals; receiving, by processing circuitry and from the sensing circuitry, one or more EEG signals generated during a first period of time; determining, by the processing circuitry and based on at least one particular feature of the one or more EEG signals generated during the first period of time, an initial indication of a cranial acute health event during the first period of time; receiving, by the processing circuitry and from the sensing circuitry, one or more EEG signals generated during a second period of time; determining, by the processing circuitry, whether a difference between the at least one particular feature of the one or more EEG signals generated during the first period of time and at least one corresponding particular feature of the one or more EEG signals generated during the second period of time satisfies a cranial acute health event mimic threshold; in response to the difference satisfying the cranial acute health event mimic threshold, determining, by the processing circuitry, the initial indication of cranial acute health event during the first period of time is a cranial acute health event mimic; and in response to the difference not satisfying the cranial acute health event mimic threshold, verifying, by the processing circuitry, the initial indication of cranial acute health event during the first period of time is a cranial acute health event.

Example 22: The method of example 21, wherein the cranial acute health event is a stroke.

Example 23: The method of any of examples 21-22, wherein the at least one particular feature comprises a bandwidth characteristic of the one or more EEG signals.

Example 24: The method of example 23, wherein the bandwidth characteristic includes a bandwidth ratio.

Example 25: The method of example 24, wherein the bandwidth ratio includes a delta-alpha ratio.

Example 26: The method of any of examples 21-25, wherein the second period of time does not overlap with the first period of time.

Example 27: The method of any of examples 21-25, wherein the second period of time is after the first period of time.

Example 28: The method of any of examples 21-27 further includes determining, based on the one or more EEG signals generated during the first period of time, the at least one particular feature of the one or more EEG signals generated during the first period of time; and determining, based on the one or more EEG signals generated during the second period of time, the at least one corresponding particular feature of the one or more EEG signals generated during the second period of time.

Example 29: The method of example 28 further includes determining the at least one corresponding particular feature of the one or more EEG signals generated during the second period at an end of the second period of time.

Example 30: The method of any of examples 21-29 further includes determining the difference satisfies the cranial acute health event mimic threshold based on a value of the difference being greater than the cranial acute health event mimic threshold.

Example 31: A method includes sensing, by sensing circuitry and via at least two electrodes of a plurality of electrodes, electrical signals from a patient; generating, by the sensing circuitry and based on the electrical signals, one or more electroencephalography (EEG) signals; receiving, by processing circuitry and from the sensing circuitry, one or more EEG signals of a first brain hemisphere; determining, by the processing circuitry and based on at least one particular feature of the one or more EEG signals of the first brain hemisphere, an initial indication of a cranial acute health event; receiving, by the processing circuitry and from the sensing circuitry, one or more EEG signals of a second brain hemisphere; determining, by the processing circuitry, whether a difference between the at least one particular feature of the one or more EEG signals of the first brain hemisphere and at least one corresponding particular feature of the one or more EEG signals of the second brain hemisphere satisfies a cranial acute health event brain hemisphere mimic threshold; in response to the difference satisfying the cranial acute health event brain hemisphere mimic threshold, determining, by the processing circuitry, the initial indication of cranial acute health event is a cranial acute health event mimic; and in response to the difference not satisfying the cranial acute health event brain hemisphere mimic threshold, verifying, by the processing circuitry, the initial indication of cranial acute health event is a cranial acute health event.

Example 32: The method of example 31, wherein the cranial acute health event is a stroke.

Example 33: The method of any of examples 31-32, wherein the at least one particular feature comprises a bandwidth characteristic of the one or more EEG signals.

Example 34: The method of example 33, wherein the bandwidth characteristic includes a bandwidth ratio.

Example 35: The method of example 34, wherein the bandwidth ratio includes a delta-alpha ratio.

Example 36: The method of any of examples 31-35 further includes determining, by the processing circuitry, the at least one particular feature of the one or more EEG signals of the first brain hemisphere; and determining, by the processing circuitry, the at least one particular feature of the one or more EEG signals of the second brain hemisphere.

Example 37: The method of example 36 further includes determining, by the processing circuitry, the at least one particular feature of the one or more EEG signals of the first brain hemisphere during a first period of time; and determining, by the processing circuitry, the at least one particular feature of the one or more EEG signals of the second brain hemisphere during a second period of time, wherein the second period of time is after the first period of time.

Example 38: The method of any of examples 31-37 further includes determining the difference satisfies the cranial acute health event brain hemisphere mimic threshold based on a value of the difference being less than the cranial acute health event brain hemisphere mimic threshold.

Example 39: The method of any of examples 31-37 further includes determining the difference does not satisfy the cranial acute health event brain hemisphere mimic threshold based on a value of the difference being greater than the cranial acute health event brain hemisphere mimic threshold.

Example 40: The method of any of examples 31-39, wherein the first brain hemisphere is a side brain hemisphere and the second brain hemisphere is the opposite side brain hemisphere.

Example 41: A computer-readable medium comprising instructions that, when executed, cause processing circuitry to execute any of the methods of examples 21-40.

Various examples have been described. These and other examples are within the scope of the following claims.

What is claimed is:

1. A system comprising:
a plurality of electrodes;
sensing circuitry configured to:
sense, via at least two electrodes of the plurality of electrodes, electrical signals from a patient; and
generate, based on the electrical signals, one or more electroencephalography (EEG) signals; and
processing circuitry configured to:
receive, from the sensing circuitry, one or more EEG signals generated during a first period of time;
determine, based on at least one particular feature of the one or more EEG signals generated during the first period of time, an initial indication of a cranial acute health event during the first period of time;
receive, from the sensing circuitry, one or more EEG signals generated during a second period of time;
determine whether a difference between the at least one particular feature of the one or more EEG signals generated during the first period of time and at least one corresponding particular feature of the one or more EEG signals generated during the second period of time satisfies a cranial acute health event mimic threshold;
in response to the difference satisfying the cranial acute health event mimic threshold, determine the initial indication of cranial acute health event during the first period of time is a cranial acute health event mimic; and
in response to the difference not satisfying the cranial acute health event mimic threshold, verify the initial indication of cranial acute health event during the first period of time is a cranial acute health event.

2. The system of claim 1, wherein the cranial acute health event is a stroke.

3. The system of claim 1, wherein the at least one particular feature comprises a bandwidth characteristic of the one or more EEG signals.

4. The system of claim 3, wherein the bandwidth characteristic includes a bandwidth ratio.

5. The system of claim 4, wherein the bandwidth ratio includes a delta-alpha ratio.

6. The system of claim 1, wherein the second period of time does not overlap with the first period of time.

7. The system of claim 1, wherein the second period of time is after the first period of time.

8. The system of claim 1, wherein the processing circuitry is further configured to:
determine, based on the one or more EEG signals generated during the first period of time, the at least one particular feature of the one or more EEG signals generated during the first period of time; and
determine, based on the one or more EEG signals generated during the second period of time, the at least one corresponding particular feature of the one or more EEG signals generated during the second period of time.

9. The system of claim 8, wherein the processing circuitry is further configured to determine the at least one corresponding particular feature of the one or more EEG signals generated during the second period at an end of the second period of time.

10. The system of claim 9, wherein the processing circuitry is further configured to determine the difference satisfies the cranial acute health event mimic threshold based on a value of the difference being greater than the cranial acute health event mimic threshold.

11. A system comprising:
a plurality of electrodes;
sensing circuitry configured to:
sense, via at least two electrodes of the plurality of electrodes, electrical signals from a patient; and
generate, based on the electrical signals, one or more electroencephalography (EEG) signals; and
processing circuitry configured to:
receive, from the sensing circuitry, one or more EEG signals of a first brain hemisphere;
determine, based on at least one particular feature of the one or more EEG signals of the first brain hemisphere, an initial indication of a cranial acute health event;
receive, from the sensing circuitry, one or more EEG signals of a second brain hemisphere;
determine whether a difference between the at least one particular feature of the one or more EEG signals of the first brain hemisphere and at least one corresponding particular feature of the one or more EEG signals of the second brain hemisphere satisfies a cranial acute health event brain hemisphere mimic threshold;
in response to the difference satisfying the cranial acute health event brain hemisphere mimic threshold, determine the initial indication of cranial acute health event is a cranial acute health event mimic; and
in response to the difference not satisfying the cranial acute health event brain hemisphere mimic threshold, verify the initial indication of cranial acute health event is a cranial acute health event.

12. The system of claim 11, wherein the cranial acute health event is a stroke.

13. The system claim 12, wherein the at least one particular feature comprises a bandwidth characteristic of the one or more EEG signals.

14. The system of claim 13, wherein the bandwidth characteristic includes a bandwidth ratio.

15. The system of claim 14, wherein the bandwidth ratio includes a delta-alpha ratio.

16. The system of claim 15, wherein the processing circuitry is further configured to:
determine the at least one particular feature of the one or more EEG signals of the first brain hemisphere; and
determine the at least one particular feature of the one or more EEG signals of the second brain hemisphere.

17. The system of claim 16, wherein the processing circuitry is further configured to:
determine the at least one particular feature of the one or more EEG signals of the first brain hemisphere during a first period of time; and
determine the at least one particular feature of the one or more EEG signals of the second brain hemisphere during a second period of time,
wherein the second period of time is after the first period of time.

18. The system of claim 11, wherein the processing circuitry is further configured to determine the difference satisfies the cranial acute health event brain hemisphere mimic threshold based on a value of the difference being less than the cranial acute health event brain hemisphere mimic threshold.

19. The system of claim 11, wherein the processing circuitry is further configured to determine the difference do not satisfy the cranial acute health event brain hemisphere mimic threshold based on a value of the difference being greater than the cranial acute health event brain hemisphere mimic threshold.

20. The system of claim 11, wherein the first brain hemisphere is a first side brain hemisphere and the second brain hemisphere is an opposite side brain hemisphere.
